(19) United States
(12) Patent Application Publication
(10) Pub. No.: US 2022/0193127 A1
(43) Pub. Date: Jun. 23, 2022

(54) IMMUNOTHERAPY FOR OPIOID ADDICTION

(71) Applicant: Therapeutic Solutions International, Inc., Oceanside, CA (US)

(72) Inventors: Thomas Ichim, Oceanside, CA (US); Timothy G. Dixon, Oceanside, CA (US); Famela Ramos, Oceanside, CA (US); Wais Kaihani, Oceanside, CA (US); James Veltmeyer, Oceanside, CA (US); Kalina O'Connor, Oceanside, CA (US)

(73) Assignee: Therapeutic Solutions International, Inc., Oceanside, CA (US)

(21) Appl. No.: 17/556,985

(22) Filed: Dec. 20, 2021

Related U.S. Application Data
(60) Provisional application No. 63/128,759, filed on Dec. 21, 2020.

(51) Int. Cl. A61K 35/16; A61P 25/36; G01N 33/68

(52) U.S. Cl. CPC A61K 35/16; G01N 33/6869; A61P 25/36

(57) ABSTRACT

Disclosed are means, methods and compositions of matter useful for reduction of brain inflammation and prevention of opioid addiction and/or tolerance. In one embodiment the invention provides utilization of platelet rich plasma (PRP), alone, or admixed with regenerative/anti-inflammatory adjuvants, for reduction of neural inflammation. In one embodiments PRP is admixed with oxytocin and administered intranasally in a patient at risk of opioid addiction. In another embodiment, PRP is admixed with fortified and non-fortified nigella sativa oil, and/or pterostilbene and administered intranasally. Other embodiments include utilization of autologous stromal vascular fraction cells alone and/or admixed with regenerative/anti-inflammatory adjuvants.

### CROSS-REFERENCE TO RELATED APPLICATIONS

This application claims priority to U.S. Provisional Application No. 63/128,759, filed Dec. 21, 2020, which is herein incorporated by reference in its entirety.

### FIELD OF THE INVENTION

The invention pertains to the field of treating opioid addiction, more specifically the invention relates to treatment of opioid addiction by administering platlet rich plasma (PRP) alone or with regenerative/anti-inflammatory adjuvants.

### BACKGROUND OF THE INVENTION

People who abuse opioid drugs typically suffers from psychological dependence which is a compulsion for the continuous administration of a drug for its euphoric effects despite any adverse effects that may occur, and include morphine or derivatives thereof, cocaine, and methamphetamine or derivatives thereof. Opioids such as morphine, which is a narcotic analgesic agent, has the psychological excitatory effects with most common symptoms of hallucinosis and delirium when used in relatively high doses or when repeatedly used for longer periods even in low doses. Such psychotoxicity occurrence is augmented by the repeated administration of morphine, and a very high physical and psychological dependence is thus developed. That is, the abused drugs have the major properties of exciting the central nervous system and increasing the psychological craving for continuous consumption of the drugs. The abused drugs increase spontaneous locomotor activity by continuous administration and induce psychological dependence thereon. It is known that the continuous administration of the abused drugs leads to the exhaustion of dopamine and reduced activity of dopamine in the nervous system. To compensate for the reduced activity of dopamine, dopamine neurons are activated. Thus, the postsynaptic dopamine receptors become hypersensitive, and spontaneous locomotor activity is increased, resulting in the development of strong craving for the continuous consumption of the drugs. Due to such a psychoactive effect of morphine, users depending on morphine have been continuously increased. Since such abuse of morphine causes severe social problems, there is an urgent need for the development of agents for treating and preventing morphine addiction.

Since previous approaches have failed to stop or substantially inhibit opioid addiction, the current patent aims to utilize immunological approaches to reduce opioid addiction. The brain-immune interaction is well described. In one series of experiments suggests that immune cells may alter brain function. For example, in one study, severe combined immune deficient (scid) mice were used to examine the role of the immune system in learning behavior of mice in a variety of cognitive tasks. While no motivation or motor function deficits were found in the immune deficient mice, there was significant impairment in acquisition of cognitive tasks as compared to wild-type (WT) control mice. Moreover, acute depletion of adaptive immunity in adult WT mice significantly impaired learning behavior. Passive transfer of autologous T cells into WT mice following ablation of adaptive immunity restored previously impaired cognitive function [1]. The notion of T cells altering mental function has also been shown by studies in which mice devoid of functional T cells are impaired in performance of cognitive tasks such as Morris water maze (MWM), Barnes maze and others. Studies have demonstrated that This is a reversible phenomenon; injection of immune deficient mice with T cells from wild type counterparts improves their cognitive function. It was further shown that that macrophages alternatively activated in vitro (M2 cells) can circumvent the need for ‘pro-cognitive’ T cells when injected intravenously into immune deficient mice. Therefore T cells, as well as type 2 macrophages appear to play a role in memory [2]. These studies, suggesting that immune cells can influence mental activities are further supported by studies in which immunization can actually reduce post stress mental anxiety [3]. Others have also reported anxiolytic [4], and memory restoring [5, 6], effects of T cells,

One of the most fundamental demonstrations of brain immune cross-talk are experiments demonstrating Pavlovian conditioning of immunity. Numerous mouse and human studies have shown feasibility of conditioning a scent or a taste with either immune suppressive [7-16], or immune stimulatory drugs [17-21]. The practical relevance of such conditioning experiments is seen in efficacy of using the conditioned stimuli alone to treat disease. For example, Bauer et al used a saccharin solution as conditioned stimulus, which was combined with the immune suppressive drug cyclosporin. After 6 pairings of cyclosporine and saccharin, animals where induced to undergo autoimmune uveitis using a standard protocol. Decreased Th1 responses, which are suppressed by cyclosporine, but not Th17, which is not suppressed by cyclosporine, was observed [22]. This, and numerous other experiments, demonstrated that conditioning of immunity by mental association can be utilized to treat pathology [23], or induce pathology [24]. An interesting experiment used a psychological technique to “amplify” the Pavlovian Conditioning effect. Luckemann et al. utilized the collagen II rheumatoid arthritis model with Pavlovian conditioning of a new taste to the rats. Saccharin, which served as the conditioned stimulus was paired with cyclosporin A. Arthritis was induced by injection of type II collagen with adjuvant. Fourteen days later, at the first occurrence of clinical symptoms, saccharin together with low-dose cyclosporine as reminder cues to prevent the conditioned response from being extinguished. The low-dose cyclosporine was below the concentration needed to induce a therapeutic effect in the animals. The arthritis score and histologic inflammatory symptoms were suppressed as much as was accomplished with full-dose pharmacologic treatment [25].

Numerous mechanisms of brain-immune interactions have been elucidated. For example, it is known that all major immune organs are highly innervated. In some experiments beta adrenergic nerves have been demonstrated to be essential for Pavlovian conditioning of immune modulation [26-28]. Interestingly, other immunological events are also related to involvement of nervous system. For example, the process of bone marrow stem cell mobilization by G-CSF administration has been shown to be dependent on sympathetic nervous system [29-33], as well as endocannabinoid receptor [34], activation in the osteal bone microenvironment. Another example of the brain-immune interplay is the prevention of sepsis associated death in animals via a vagus-nerve dependent manner, in which stimulation of the nerve is protective and inactivation is associated with hypersensitivity to toxicity [35-38]. The technology of vagus nerve stimulation is used to treat epilepsy in humans. Since commercially available devices exist that are clinically applicable, one study sought to determine whether stimulation of this nerve could be clinically utilized to treated rheumatoid arthritis. In a clinical study it was demonstrated that an implantable vagus nerve-stimulating device in epilepsy patients inhibits peripheral blood production of TNF, IL-1(3, and IL-6. Furthermore, it was demonstrated that vagus nerve stimulation arthritis patients significantly inhibited TNF production for up to 84 d. Moreover, RA disease severity, as measured by standardized clinical composite scores, improved significantly [39]. Besides this pilot trial, there are no real evidence of clinical translation using the nervous system to augment the immune system. At a preclinical level, there are less studies looking at whether the immune system can alter the nervous system.

### SUMMARY

1. A 1^{st} embodiment includes a method of reducing opioid addiction comprising the steps of: a) obtaining a patient with a propensity for opioid addiction; b) assessing one or more markers associated with said opioid addiction; c) administering to said patient platelet rich plasma and/or cord blood plasma alone or in combination with an anti-inflammatory/regenerative adjuvant; d) assessing levels of said one or more markers associated with said opioid addiction; and e) adjusting dosage/frequency of administration of said platelet rich plasma alone or in combination with an anti-inflammatory/regenerative adjuvant.

2. The method of embodiment 1, wherein propensity for opioid addiction is associated with augmentation of inflammatory cytokines and/or mediators in a biological fluid as compared to an age-matched control.

3. The method of embodiment 2, wherein said biological fluid is blood, plasma, or serum.

4. The method of embodiment 2, wherein said biological fluid is urine.

5. The method of embodiment 2, wherein said biological fluid is saliva.

6. The method of embodiment 2, wherein said biological fluid is tears.

7. The method of embodiment 2, wherein said biological fluid is bronchiolar lavage fluid.

8. The method of embodiment 2, wherein said biological fluid is cerebral spinal fluid.

9. The method of embodiment 2, wherein said inflammatory cytokine is IL-1 beta.

10. The method of embodiment 2, wherein said inflammatory cytokine is IL-6.

11. The method of embodiment 2, wherein said inflammatory cytokine is IL-7.

12. The method of embodiment 2, wherein said inflammatory cytokine is IL-8.

13. The method of embodiment 2, wherein said inflammatory cytokine is IL-12.

14. The method of embodiment 2, wherein said inflammatory cytokine is IL-15.

15. The method of embodiment 2, wherein said inflammatory cytokine is IL-17.

16. The method of embodiment 2, wherein said inflammatory cytokine is IL-18.

17. The method of embodiment 2, wherein said inflammatory cytokine is IL-21.

18. The method of embodiment 2, wherein said inflammatory cytokine is IL-9.

19. The method of embodiment 2, wherein said inflammatory cytokine is IL-27.

20. The method of embodiment 2, wherein said inflammatory cytokine is IL-23.

21. The method of embodiment 2, wherein said inflammatory cytokine is IL-33.

22. The method of embodiment 2, wherein said inflammatory cytokine is BAFF.

23. The method of embodiment 2, wherein said inflammatory cytokine is 4-1 BBL.

23. The method of embodiment 2, wherein said inflammatory cytokine is TNFSF8.

24. The method of embodiment 2, wherein said inflammatory cytokine is CD40LG.

25. The method of embodiment 2, wherein said inflammatory cytokine is CD70.

26. The method of embodiment 2, wherein said inflammatory cytokine is CD95L.

27. The method of embodiment 2, wherein said inflammatory cytokine is TNFSF8.

28. The method of embodiment 2, wherein said inflammatory cytokine is EDA-A1.

29. The method of embodiment 2, wherein said inflammatory cytokine is TNFSF15.

30. The method of embodiment 2, wherein said inflammatory cytokine is TNF-beta.

31. The method of embodiment 2, wherein said inflammatory cytokine is LTB.

32. The method of embodiment 2, wherein said inflammatory cytokine is TNF-alpha.

33. The method of embodiment 2, wherein said inflammatory cytokine is TNFSF10.

34. The method of embodiment 2, wherein said inflammatory cytokine is TNFSF11.

35. The method of embodiment 2, wherein said inflammatory cytokine is TNFSF12.

36. The method of embodiment 2, wherein said inflammatory cytokine is TNFSF13.

37. The method of embodiment 2, wherein said inflammatory cytokine is TNFSF4.

38. The method of embodiment 2, wherein said inflammatory cytokine is TNFSF10.

39. The method of embodiment 2, wherein said inflammatory cytokine is IFNA1.

40. The method of embodiment 2, wherein said inflammatory cytokine is IFNA10.

41. The method of embodiment 2, wherein said inflammatory cytokine is IFNA13.

42. The method of embodiment 2, wherein said inflammatory cytokine is IFNA14.

43. The method of embodiment 2, wherein said inflammatory cytokine is IFNA2.

44. The method of embodiment 2, wherein said inflammatory cytokine is IFNA4.

45. The method of embodiment 2, wherein said inflammatory cytokine is IFNA7.

46. The method of embodiment 2, wherein said inflammatory cytokine is IFNB1.

47. The method of embodiment 2, wherein said inflammatory cytokine is IFNE.

48. The method of embodiment 2, wherein said inflammatory cytokine is IFNG.

49. The method of embodiment 2, wherein said inflammatory cytokine is IFNZ.

50. The method of embodiment 2, wherein said inflammatory cytokine is IFNA8.

51. The method of embodiment 2, wherein said inflammatory cytokine is IFNA5.

52. The method of embodiment 2, wherein said inflammatory cytokine is IFNW1.

53. The method of embodiment 2, wherein said inflammatory cytokine is CLCF1.

54. The method of embodiment 2, wherein said inflammatory cytokine is CNTF.

55. The method of embodiment 2, wherein said inflammatory cytokine is IFNW1.

56. The method of embodiment 2, wherein said inflammatory cytokine is IL-11.

57. The method of embodiment 2, wherein said inflammatory cytokine is IL-31.

58. The method of embodiment 2, wherein said inflammatory cytokine is leptin.

59. The method of embodiment 2, wherein said inflammatory cytokine is LIF.

60. The method of embodiment 2, wherein said inflammatory cytokine is OSM.

61. The method of embodiment 2, wherein said inflammatory cytokine is IL-1A.

62. The method of embodiment 2, wherein said inflammatory cytokine is IL-1B.

63. The method of embodiment 2, wherein said inflammatory cytokine is IL1F10.

64. The method of embodiment 2, wherein said inflammatory cytokine is IL1F3.

65. The method of embodiment 2, wherein said inflammatory cytokine is IL1F5.

66. The method of embodiment 2, wherein said inflammatory cytokine is IL-1A.

67. The method of embodiment 2, wherein said inflammatory cytokine is ILF6.

68. The method of embodiment 2, wherein said inflammatory cytokine is IL1F8.

69. The method of embodiment 2, wherein said inflammatory cytokine is IL1RL2.

70. The method of embodiment 2, wherein said inflammatory cytokine is IL1F9.

71. The method of embodiment 2, wherein said inflammatory mediator is a chemokine.

72. The method of embodiment 72, wherein said chemokine is CCL1.

73. The method of embodiment 72, wherein said chemokine is CCL11.

74. The method of embodiment 72, wherein said chemokine is CCL12.

75. The method of embodiment 72, wherein said chemokine is MCP4.

76. The method of embodiment 72, wherein said chemokine is CCL14.

77. The method of embodiment 72, wherein said chemokine is CCL15.

72. The method of embodiment 72, wherein said chemokine is CCL16.

73. The method of embodiment 72, wherein said chemokine is TARC.

74. The method of embodiment 72, wherein said chemokine is CCL18.

75. The method of embodiment 72, wherein said chemokine is CCL19.

76. The method of embodiment 72, wherein said chemokine is MCP1.

77. The method of embodiment 72, wherein said chemokine is CCL20.

78. The method of embodiment 72, wherein said chemokine is CCL21.

79. The method of embodiment 72, wherein said chemokine is MDC.

80. The method of embodiment 72, wherein said chemokine is CCL23.

81. The method of embodiment 72, wherein said chemokine is CCL24.

82. The method of embodiment 72, wherein said chemokine is CCL25.

83. The method of embodiment 72, wherein said chemokine is CCL26.

84. The method of embodiment 72, wherein said chemokine is CCL27.

85. The method of embodiment 72, wherein said chemokine is CCL28.

86. The method of embodiment 72, wherein said chemokine is CCL3.

87. The method of embodiment 72, wherein said chemokine is CCL3L3.

88. The method of embodiment 72, wherein said chemokine is CCL4.

89. The method of embodiment 72, wherein said chemokine is CCL1.

90. The method of embodiment 72, wherein said chemokine is LAG-1.

91. The method of embodiment 72, wherein said chemokine is CCL5.

92. The method of embodiment 72, wherein said chemokine is CCL6.

93. The method of embodiment 72, wherein said chemokine is CCL7.

94. The method of embodiment 72, wherein said chemokine is CCL8.

95. The method of embodiment 72, wherein said chemokine is CCL9.

96. The method of embodiment 72, wherein said chemokine is CX3CL1.

97. The method of embodiment 72, wherein said chemokine is CXCL1.

98. The method of embodiment 72, wherein said chemokine is CXCL10.

99. The method of embodiment 72, wherein said chemokine is CXCL11.

100. The method of embodiment 72, wherein said chemokine is CXCL12.

101. The method of embodiment 72, wherein said chemokine is CXCL13.

102. The method of embodiment 72, wherein said chemokine is CXCL14.

103. The method of embodiment 72, wherein said chemokine is CXCL15.

104. The method of embodiment 72, wherein said chemokine is CXCL16.

105. The method of embodiment 72, wherein said chemokine is CXCL17.

106. The method of embodiment 72, wherein said chemokine is MIP-2.

107. The method of embodiment 72, wherein said chemokine is CXCL3.

108. The method of embodiment 72, wherein said chemokine is CXCL4.

109. The method of embodiment 72, wherein said chemokine is CXCL5.

110. The method of embodiment 72, wherein said chemokine is CXCL6.

111. The method of embodiment 72, wherein said chemokine is Ppbp.

112. The method of embodiment 72, wherein said chemokine is CXCL9.

113. The method of embodiment 72, wherein said chemokine is XCL1.

114. The method of embodiment 72, wherein said chemokine is XCL2.

115. The method of embodiment 72, wherein said chemokine is FAM19A1.

116. The method of embodiment 72, wherein said chemokine is FAM19A2.

117. The method of embodiment 72, wherein said chemokine is FAM19A3.

118. The method of embodiment 72, wherein said chemokine is FAM19A4.

119. The method of embodiment 72, wherein said chemokine is FAM19A5.

120. The method of embodiment 2, wherein said inflammatory mediator is kynurenin.

121. The method of embodiment 2, wherein said inflammatory mediator is spermidine/spermine N1-acetyltransferase 1 (SAT1).

122. The method of embodiment 2, wherein said inflammatory mediator is forkhead box N3 (FOXN3).

123. The method of embodiment 2, wherein said inflammatory mediator is guanylate binding protein 1 (GBP1).

124. The method of embodiment 2, wherein said inflammatory mediator is phosphoinositide-3-kinase regulatory subunit 5 (PIK3R5).

125. The method of embodiment 2, wherein said inflammatory mediator is apolipoprotein L2 (APOL2).

126. The method of embodiment 2, wherein said inflammatory mediator is ATPase H+ transporting lysosomal 9 kDa, VO subunit el (ATP6VOE1).

127. The method of embodiment 2, wherein said inflammatory mediator is GRINL1A complex locus (GCOM1).

128. The method of embodiment 2, wherein said inflammatory mediator is lipoma HMGIC fusion partner (LHFP).

129. The method of embodiment 2, wherein said inflammatory mediator is lipase A (LIPA).

130. The method of embodiment 2, wherein said inflammatory mediator is myristoylated alanine-rich protein kinase C substrate (MARCKS).

131. The method of embodiment 2, wherein said inflammatory mediator is 6-phosphogluconolactonase (PGLS).

132. The method of embodiment 2, wherein said inflammatory mediator is phosphatase and tensin homolog (PTEN).

133. The method of embodiment 2, wherein said inflammatory mediator is reversion-inducing-cysteine-rich protein with kazal motifs (RECK).

134. The method of embodiment 2, wherein said inflammatory mediator is ABCB1.

135. The method of embodiment 2, wherein said inflammatory mediator is ACACB.

136. The method of embodiment 2, wherein said inflammatory mediator is ACAT1.

137. The method of embodiment 2, wherein said inflammatory mediator is ACHE.

138. The method of embodiment 2, wherein said inflammatory mediator is ADRB1.

139. The method of embodiment 2, wherein said inflammatory mediator is ADRB2.

140. The method of embodiment 2, wherein said inflammatory mediator is AKT1.

141. The method of embodiment 2, wherein said inflammatory mediator is AKT2.

142. The method of embodiment 2, wherein said inflammatory mediator is ANGPT1.

143. The method of embodiment 2, wherein said inflammatory mediator is APOB.

144. The method of embodiment 2, wherein said inflammatory mediator is APOH.

145. The method of embodiment 2, wherein said inflammatory mediator is APOL3.

146. The method of embodiment 2, wherein said inflammatory mediator is APOL4

147. The method of embodiment 2, wherein said inflammatory mediator is AVEN.

148. The method of embodiment 2, wherein said inflammatory mediator is CETP.

149. The method of embodiment 2, wherein said inflammatory mediator is CHAT.

150. The method of embodiment 2, wherein said inflammatory mediator is CHKB.

151. The method of embodiment 2, wherein said inflammatory mediator is CPT1A.

152. The method of embodiment 2, wherein said inflammatory mediator is CRHR2.

153. The method of embodiment 2, wherein said inflammatory mediator is DBH.

154. The method of embodiment 2, wherein said inflammatory mediator is DRD3.

155. The method of embodiment 2, wherein said inflammatory mediator is DRD4.

156. The method of embodiment 2, wherein said inflammatory mediator is DRD5.

157. The method of embodiment 2, wherein said inflammatory mediator is DTNBP1.

158. The method of embodiment 2, wherein said inflammatory mediator is F1132252.

159. The method of embodiment 2, wherein said inflammatory mediator is FLT1.

160. The method of embodiment 2, wherein said inflammatory mediator is GABRA2.

161. The method of embodiment 2, wherein said inflammatory mediator is GAL.

162. The method of embodiment 2, wherein said inflammatory mediator is GNAO1.

163. The method of embodiment 2, wherein said inflammatory mediator is GYS2.

163. The method of embodiment 2, wherein said inflammatory mediator is ABCA1.

164. The method of embodiment 2, wherein said inflammatory mediator is F1110357.

165. The method of embodiment 2, wherein said inflammatory mediator is CASC1.

166. The method of embodiment 2, wherein said inflammatory mediator is DHR9.

167. The method of embodiment 2, wherein said inflammatory mediator is DISC1.

168. The method of embodiment 2, wherein said inflammatory mediator is EIF2AK2.

169. The method of embodiment 2, wherein said inflammatory mediator is MAP3K3.

170. The method of embodiment 2, wherein said inflammatory mediator is MT-ND6.

171. The method of embodiment 2, wherein said inflammatory mediator is RBM-47.

172. The method of embodiment 2, wherein said inflammatory mediator is RPTOR.

173. The method of embodiment 2, wherein said inflammatory mediator is RICTOR.

174. The method of embodiment 2, wherein said inflammatory mediator is SAMD9L.

175. The method of embodiment 2, wherein said inflammatory mediator is SCARF-1.

176. The method of embodiment 2, wherein said inflammatory mediator is SLC36A1.

177. The method of embodiment 2, wherein said inflammatory mediator is STAT1.

178. The method of embodiment 2, wherein said inflammatory mediator is COX5B.

179. The method of embodiment 2, wherein said inflammatory mediator is SMARCA1.

180. The method of embodiment 2, wherein said inflammatory mediator is UBA6.

181. The method of embodiment 2, wherein said inflammatory mediator is ZC3HAV1.

182. The method of embodiment 2, wherein said inflammatory mediator is TNK2.

183. The method of embodiment 2, wherein said inflammatory mediator is CD24.

184. The method of embodiment 2, wherein said inflammatory mediator is ATP13A2.

185. The method of embodiment 2, wherein said inflammatory mediator is EPHX1.

186. The method of embodiment 2, wherein said inflammatory mediator is HTRA1.

187. The method of embodiment 2, wherein said inflammatory mediator is LEPR.

188. The method of embodiment 2, wherein said inflammatory mediator is SPTBN1.

189. The method of embodiment 2, wherein said inflammatory mediator is MBNL2.

190. The method of embodiment 2, wherein said inflammatory mediator is OR2J3.

191. The method of embodiment 2, wherein said inflammatory mediator is RHEB.

192. The method of embodiment 2, wherein said inflammatory mediator is GRINA.

193. The method of embodiment 2, wherein said inflammatory mediator is KCNJ2.

194. The method of embodiment 2, wherein said inflammatory mediator is TOP1.

195. The method of embodiment 2, wherein said inflammatory mediator is GYS2.

196. The method of embodiment 2, wherein said inflammatory mediator is ICAM1.

197. The method of embodiment 2, wherein said inflammatory mediator is INSR.

198. The method of embodiment 2, wherein said inflammatory mediator is KDR.

199. The method of embodiment 2, wherein said inflammatory mediator is LDLR.

200. The method of embodiment 2, wherein said inflammatory mediator is LIPE.

201. The method of embodiment 2, wherein said inflammatory mediator is LIPF.

202. The method of embodiment 2, wherein said inflammatory mediator is LOC391530.

203. The method of embodiment 2, wherein said inflammatory mediator is OLR1.

204. The method of embodiment 2, wherein said inflammatory mediator is OXT.

205. The method of embodiment 2, wherein said inflammatory mediator is PIK3C2G.

206. The method of embodiment 2, wherein said inflammatory mediator is PIK3C3.

207. The method of embodiment 2, wherein said inflammatory mediator is PIK3R1.

208. The method of embodiment 2, wherein said inflammatory mediator is PPARG.

209. The method of embodiment 2, wherein said inflammatory mediator is PRKAA1.

210. The method of embodiment 2, wherein said inflammatory mediator is PRKAB 1.

211. The method of embodiment 2, wherein said inflammatory mediator is RARB.

212. The method of embodiment 2, wherein said inflammatory mediator is RARG.

213. The method of embodiment 2, wherein said inflammatory mediator is RXRA.

214. The method of embodiment 2, wherein said inflammatory mediator is SCARB2.

215. The method of embodiment 2, wherein said inflammatory mediator is SELE.

216. The method of embodiment 2, wherein said inflammatory mediator is SSTR3.

217. The method of embodiment 2, wherein said inflammatory mediator is leukotriene B4.

218. The method of embodiment 2, wherein said inflammatory mediator is Decanoylcarnitine.

219. The method of embodiment 2, wherein said inflammatory mediator is Butenylcarnitine.

220. The method of embodiment 2, wherein said inflammatory mediator is hydroxybutyrylcarnitine.

221. The method of embodiment 2, wherein said inflammatory mediator is 2-aminooctanoate.

222. The method of embodiment 2, wherein said inflammatory mediator is hexadecanedioate.

223. The method of embodiment 2, wherein said inflammatory mediator is 3-hydroxybutyrate.

224. The method of embodiment 2, wherein said inflammatory mediator is stearate (18:0).

225. The method of embodiment 2, wherein said inflammatory mediator is oleate (18:1n9).

226. The method of embodiment 2, wherein said inflammatory mediator is 10-heptadecenoate (17:1n7).

227. The method of embodiment 2, wherein said inflammatory mediator is 10-nonadecenoate (19:1n9).

228. The method of embodiment 2, wherein said inflammatory mediator is margarate (17:0).

229. The method of embodiment 2, wherein said inflammatory mediator is palmitate (16:0).

230. The method of embodiment 2, wherein said inflammatory mediator is arachidate (20:0).

231. The method of embodiment 2, wherein said inflammatory mediator is lysoPC a C26:0.

232. The method of embodiment 2, wherein said inflammatory mediator is lysoPC a C18:2.

233. The method of embodiment 2, wherein said inflammatory mediator is lysoPC a C18:1.

234. The method of embodiment 2, wherein said inflammatory mediator is 2-linoleoylglycerophosphoethanolamine.

235. The method of embodiment 2, wherein said inflammatory mediator is 2-1-oleoylglycerophosphoinositol.

236. The method of embodiment 2, wherein said inflammatory mediator is 2-linoleoylglycerophosphocholine.

237. The method of embodiment 2, wherein said inflammatory mediator is 1-palmitoylglycerophosphoinositol.

238. The method of embodiment 2, wherein said inflammatory mediator is 1-linoleoylglycerophosphoethanolamine.

239. The method of embodiment 2, wherein said inflammatory mediator is 2-palmitoleoylglycerophosphocholine.

240. The method of embodiment 2, wherein said inflammatory mediator is 1-arachidonoylglycerophosphoethanolamine.

241. The method of embodiment 2, wherein said inflammatory mediator is 1-linolenoylglycerophosphocholine (18:3n3).

242. The method of embodiment 2, wherein said inflammatory mediator is 1-docosapentaenoylglycerophosphocholine (22:5n3).

243. The method of embodiment 2, wherein said inflammatory mediator is 5-dodecenoate (12:1n7).

244. The method of embodiment 2, wherein said inflammatory mediator is glycerophosphorylcholine (GPC).

245. The method of embodiment 2, wherein said inflammatory mediator is dihomo-linoleate (20:2n6).

246. The method of embodiment 2, wherein said inflammatory mediator is docosadienoate (22:2n6).

247. The method of embodiment 2, wherein said inflammatory mediator is taurocholenate sulfate.

248. The method of embodiment 2, wherein said inflammatory mediator is dihomo-linoleate (20:2n6).

249. The method of embodiment 2, wherein said inflammatory mediator is sphingosine.

250. The method of embodiment 2, wherein said inflammatory mediator is SM C18:0.

251. The method of embodiment 2, wherein said inflammatory mediator is SM C24:1.

252. The method of embodiment 2, wherein said inflammatory mediator is SM (OH) C14:1.

253. The method of embodiment 2, wherein said inflammatory mediator is SM C24:0.

254. The method of embodiment 2, wherein said inflammatory mediator is cortisol.

255. The method of embodiment 2, wherein said inflammatory mediator is creatine.

256. The method of embodiment 2, wherein said inflammatory mediator is glutamine.

257. The method of embodiment 2, wherein said inflammatory mediator is glutamate.

258. The method of embodiment 2, wherein said inflammatory mediator is serine.

259. The method of embodiment 2, wherein said inflammatory mediator is Glycine.

260. The method of embodiment 2, wherein said inflammatory mediator is betaine.

261. The method of embodiment 2, wherein said inflammatory mediator is 3-hydroxyisobutyrate.

262. The method of embodiment 2, wherein said inflammatory mediator is valine.

263. The method of embodiment 2, wherein said inflammatory mediator is 3-methyl-2-oxobutyrate.

264. The method of embodiment 2, wherein said inflammatory mediator is alpha-hydroxyisovalerate.

265. The method of embodiment 2, wherein said inflammatory mediator is 3-methylglutarylcarnitine (C6).

266. The method of embodiment 2, wherein said inflammatory mediator is alpha-Aminoadipic acid.

267. The method of embodiment 2, wherein said inflammatory mediator is pipecolate.

268. The method of embodiment 2, wherein said inflammatory mediator is methionine.

269. The method of embodiment 2, wherein said inflammatory mediator is 2-aminobutyrate.

270. The method of embodiment 2, wherein said inflammatory mediator is 3-(4-hydroxyphenyl)lactate

271. The method of embodiment 2, wherein said inflammatory mediator is indolepropionate.

272. The method of embodiment 2, wherein said inflammatory mediator is C-glycosyltryptophan.

273. The method of embodiment 2, wherein said inflammatory mediator is citrulline.

274. The method of embodiment 2, wherein said inflammatory mediator is ornithine.

275. The method of embodiment 2, wherein said inflammatory mediator is bilirubin.

276. The method of embodiment 2, wherein said inflammatory mediator is biliverdin.

277. The method of embodiment 2, wherein said inflammatory mediator is N1-Methyl-2-pyridone-5-carboxamide.

278. The method of embodiment 2, wherein said inflammatory mediator is hypoxanthine.

279. The method of embodiment 2, wherein said inflammatory mediator is adenosine 5′-monophosphate (AMP).

278. The method of embodiment 2, wherein said inflammatory mediator is N2,N2-dimethylguanosine.

278. The method of embodiment 2, wherein said inflammatory mediator is uridine.

279. The method of embodiment 2, wherein said inflammatory mediator is pseudouridine.

280. The method of embodiment 2, wherein said inflammatory mediator is 5-methyluridine (ribothymidine).

281. The method of embodiment 2, wherein said inflammatory mediator is gamma-glutamyltyrosine.

282. The method of embodiment 2, wherein said inflammatory mediator is gamma-glutamylmethionine.

283. The method of embodiment 2, wherein said inflammatory mediator is bradykinin.

284. The method of embodiment 2, wherein said inflammatory mediator is HWESASXX.

285. The method of embodiment 2, wherein said inflammatory mediator is bradykinin, des-arg(9).

286. The method of embodiment 2, wherein said inflammatory mediator is bradykinin, hydroxy-pro(3).

287. The method of embodiment 2, wherein said inflammatory mediator is CRP.

288. The method of embodiment 2, wherein said inflammatory mediator is lactate.

289. The method of embodiment 2, wherein said inflammatory mediator is pyruvate.

290. The method of embodiment 2, wherein said inflammatory mediator is hypoxanthine.

291. The method of embodiment 2, wherein said inflammatory mediator is 1-methyl-2-piperidinecarboxylic acid.

292. The method of embodiment 1, wherein propensity for opioid addiction is quantified using a psychological scale.

293. The method of embodiment 292, wherein said psychological scale is a standard scale currently utilized in routine psychological practice.

294. The method of embodiment 293, wherein said scale is a test such as the Rorschach or Sentence Completion test.

295. The method of embodiment 293, wherein said scale is a personality inventory test such as the NEO-R test.

296. The method of embodiment 293, wherein said scale is the Millon Clinical Multiaxial Inventory (MCMI) scale.

297. The method of embodiment 293, wherein said scale is the Hamilton scale for depression.

298. The method of embodiment 293, wherein said scale is the Beck Hopelessness Scale.

299. The method of embodiment 1, wherein propensity for opioid addiction is quantified by assessment of numbers of circulating endothelial progenitor cells in the blood, wherein a decreased number of endothelial progenitor cells in a patient, compared to an age matched control is indicative of opioid addiction risk.

300. The method of embodiment 299, wherein said endothelial progenitor cells are capable of forming endothelial colonies when plated in vitro.

301. The method of embodiment 299, wherein said endothelial progenitor cells are capable of forming endothelial tubes when plated in a semisolid media.

302. The method of embodiment 299, wherein said endothelial progenitor cells express CD34.

303. The method of embodiment 299, wherein said endothelial progenitor cells express CD133.

304. The method of embodiment 299, wherein said endothelial progenitor cells express stem cell factor receptor.

305. The method of embodiment 299, wherein said endothelial progenitor cells express VEGF-receptor.

306. The method of embodiment 299, wherein said endothelial progenitor cells express c-met.

307. The method of embodiment 299, wherein said endothelial progenitor cells express IL-3 receptor.

308. The method of embodiment 299, wherein said endothelial progenitor cells express TNF-alpha receptor p55.

309. The method of embodiment 299, wherein said endothelial progenitor cells express TNF-alpha receptor p75.

310. The method of embodiment 299, wherein said endothelial progenitor cells express nerve growth factor receptor.

311. The method of embodiment 299, wherein said endothelial progenitor cells express TNF-alpha receptor p55.

312. The method of embodiment 299, wherein said endothelial progenitor cells express VE-Cadherin.

313. The method of embodiment 299, wherein said endothelial progenitor cells express CD31.

314. The method of embodiment 299, wherein said endothelial progenitor cells express CD117.

315. The method of embodiment 299, wherein said endothelial progenitor cells express CXCR4.

316. The method of embodiment 299, wherein said endothelial progenitor cells express CD146.

317. The method of embodiment 299, wherein said endothelial progenitor cells express PLVAP.

318. The method of embodiment 299, wherein said endothelial progenitor cells express S1P1/EDG-1.

319. The method of embodiment 299, wherein said endothelial progenitor cells express S1P2/EDG-5.

320. The method of embodiment 299, wherein said endothelial progenitor cells express S1P3/EDG-3.

321. The method of embodiment 299, wherein said endothelial progenitor cells express S1P4/EDG-6.

322. The method of embodiment 299, wherein said endothelial progenitor cells express S1P5/EDG-8.

323. The method of embodiment 299, wherein said endothelial progenitor cells express E-Selectin/CD62E.

324. The method of embodiment 299, wherein said endothelial progenitor cells express E-Selectin/CD62P.

325. The method of embodiment 299, wherein said endothelial progenitor cells express Tie-2.

326. The method of embodiment 299, wherein said endothelial progenitor cells express VCAM-1/CD106.

327. The method of embodiment 1, wherein propensity towards opioid addiction is associated with increased number of inflammatory T cells.

328. The method of embodiment 327, wherein said inflammatory T cells are T cells capable of activating macrophages.

329. The method of embodiment 328, wherein said T cells capable of activating said macrophages secrete interferon gamma, wherein said interferon gamma is involved in activation of said macrophages.

330. The method of embodiment 328, wherein said T cells capable of activating said macrophages secrete interleukin-1, wherein said interleukin-1 is involved in activation of said macrophages.

331. The method of embodiment 328, wherein said T cells capable of activating said macrophages secrete TNF-alpha, wherein said TNF-alpha is involved in activation of said macrophages.

332. The method of embodiment 328, wherein said T cells capable of activating said macrophages secrete interleukin-6, wherein said interleukin-6 is involved in activation of said macrophages.

333. The method of embodiment 328, wherein said T cells capable of activating said macrophages secrete interleukin-8, wherein said interleukin-8 is involved in activation of said macrophages.

334. The method of embodiment 328, wherein said T cells capable of activating said macrophages secrete interleukin-9, wherein said interleukin-9 is involved in activation of said macrophages.

335. The method of embodiment 328, wherein said T cells capable of activating said macrophages secrete interleukin-11, wherein said interleukin-11 is involved in activation of said macrophages.

336. The method of embodiment 328, wherein said T cells capable of activating said macrophages secrete interleukin-12, wherein said interleukin-12 is involved in activation of said macrophages.

337. The method of embodiment 328, wherein said T cells capable of activating said macrophages secrete interleukin-15, wherein said interleukin-15 is involved in activation of said macrophages.

338. The method of embodiment 328, wherein said T cells capable of activating said macrophages secrete interleukin-18, wherein said interleukin-18 is involved in activation of said macrophages.

339. The method of embodiment 328, wherein said T cells capable of activating said macrophages secrete interleukin-17, wherein said interleukin-17 is involved in activation of said macrophages.

340. The method of embodiment 328, wherein said T cells capable of activating said macrophages secrete interleukin-21, wherein said interleukin-21 is involved in activation of said macrophages.

341. The method of embodiment 328, wherein said T cells capable of activating said macrophages secrete interleukin-22, wherein said interleukin-22 is involved in activation of said macrophages.

342. The method of embodiment 328, wherein said T cells capable of activating said macrophages secrete interleukin-23, wherein said interleukin-23 is involved in activation of said macrophages.

343. The method of embodiment 328, wherein said T cells capable of activating said macrophages secrete interleukin-27, wherein said interleukin-27 is involved in activation of said macrophages.

344. The method of embodiment 328, wherein said T cells capable of activating said macrophages secrete interleukin-33, wherein said interleukin-33 is involved in activation of said macrophages.

345. The method of embodiment 328, wherein said T cells capable of activating said macrophages secrete VIP-1, wherein said VIP-1 is involved in activation of said macrophages.

346. The method of embodiment 328, wherein said T cells capable of activating said macrophages secrete PACAP, wherein said PACAP is involved in activation of said macrophages.

347. The method of embodiment 328, wherein said inflammatory T cells are Th17 cells.

348. The method of embodiment 347, wherein said Th17 cells express the transcription factor RoR gamma.

349. The method of embodiment 347, wherein said Th17 cells express angiogenic markers.

350. The method of embodiment 349, wherein said angiogenic marker is Plexin Dl.

351. The method of embodiment 347, wherein said Th17 cells express markers associated with metastasis.

352. The method of embodiment 351, wherein said metastasis associated marker is CD82.

353. The method of embodiment 347, wherein said Th17 cells express markers associated with inflammation.

354. The method of embodiment 353, wherein said marker associated with inflammation is IL-1 RAcP.

355. The method of embodiment 353, wherein said marker associated with inflammation is IL-18 receptor alpha.

356. The method of embodiment 353, wherein said marker associated with inflammation is TNF-alpha receptor p55.

357. The method of embodiment 353, wherein said marker associated with inflammation is CTLA-4.

358. The method of embodiment 353, wherein said marker associated with inflammation is BTLA-4.

359. The method of embodiment 353, wherein said marker associated with inflammation is IL-1 receptor antagonist.

360. The method of embodiment 353, wherein said marker associated with inflammation is CD144

361. The method of embodiment 347, wherein said Th17 cells express developmental associated markers.

362. The method of embodiment 361, wherein said development associated marker is Notch-1.

363. The method of embodiment 361, wherein said development associated marker is DLL3.

364. The method of embodiment 361, wherein said development associated marker is SSEA3.

365. The method of embodiment 361, wherein said development associated marker is SSEA4.

366. The method of embodiment 361, wherein said development associated marker is Nanog.

367. The method of embodiment 361, wherein said development associated marker is Sox-2.

368. The method of embodiment 361, wherein said development associated marker is Notch-1.

369. The method of embodiment 361, wherein said development associated marker is FGF-1 receptor.

370. The method of embodiment 361, wherein said development associated marker is frizzled.

371. The method of embodiment 361, wherein said development associated marker is FGF-2 receptor.

372. The method of embodiment 361, wherein said development associated marker is FGF-5.

373. The method of embodiment 361, wherein said development associated marker is PDGF-AA receptor.

374. The method of embodiment 361, wherein said development associated marker is PDGF-BB receptor.

375. The method of embodiment 361, wherein said development associated marker is EGF-1 receptor.

376. The method of embodiment 347, wherein said Th17 cells express markers associated with axon growth.

377. The method of embodiment 376, wherein said marker is Sema 7A.

378. The method of embodiment 376, wherein said marker is Sema 4D.

379. The method of embodiment 347, wherein said Th17 cells express lymphocyte markers.

380. The method of embodiment 379, wherein said lymphocyte marker is CD4.

381. The method of embodiment 379, wherein said lymphocyte marker is CD43.

382. The method of embodiment 379, wherein said lymphocyte marker is CD99.

383. The method of embodiment 379, wherein said lymphocyte marker is NTB-A.

384. The method of embodiment 379, wherein said lymphocyte marker is LAIR1.

385. The method of embodiment 379, wherein said lymphocyte marker is CCRL2.

386. The method of embodiment 347, wherein said Th17 cells express markers found on myeloid cells.

387. The method of embodiment 386, wherein said myeloid marker found on Th17 cells is CD200.

388. The method of embodiment 347, wherein said Th17 cells express markers found on thrombocytes.

389. The method of embodiment 388, wherein said markers found on thrombocytes is CD49f.

390. The method of embodiment 347, wherein said Th17 cells express markers found on erythrocytes.

391. The method of embodiment 390, wherein said marker found on erythrocytes is TRA-1-85.

392. The method of embodiment 347, wherein said Th17 cells express TfR.

393. The method of embodiment 347, wherein said Th17 cells express A33.

394. The method of embodiment 347, wherein said Th17 cells express adhesion and/or signaling molecules.

395. The method of embodiment 394, wherein said adhesion and/or signaling molecules found on Th17 cells are CD97.

396. The method of embodiment 394, wherein said adhesion and/or signaling molecules found on Th17 cells are CD53.

397. The method of embodiment 394, wherein said adhesion and/or signaling molecules found on Th17 cells are DEP-1.

398. The method of embodiment 394, wherein said adhesion and/or signaling molecules found on Th17 cells are ALCAM.

399. The method of embodiment 394, wherein said adhesion and/or signaling molecules found on Th17 cells are DNAM-1.

400. The method of embodiment 394, wherein said adhesion and/or signaling molecules found on Th17 cells are CD48.

401. The method of embodiment 394, wherein said adhesion and/or signaling molecules found on Th17 cells are IGF-II receptor

402. The method of embodiment 394, wherein said adhesion and/or signaling molecules found on Th17 cells are LRP-6.

403. The method of embodiment 1, wherein said marker associated with opioid addiction is a reduction in T regulatory cells.

404. The method of embodiment 403, wherein said T regulatory cell is a cell expressing a transcription factor capable of inducing a suppressive phenotype upon T cells.

405. The method of embodiment 404, wherein said transcription factor is FoxP3.

406. The method of embodiment 403, wherein said T regulatory cell expresses CD4.

407. The method of embodiment 403, wherein said T regulatory cell expresses CD25.

408. The method of embodiment 403, wherein said T regulatory cell expresses Helios.

409. The method of embodiment 403, wherein said T regulatory cell expresses GITR ligand.

410. The method of embodiment 403, wherein said T regulatory cell expresses membrane bound TGF-beta.

411. The method of embodiment 403, wherein said T regulatory cell expresses Fas ligand.

412. The method of embodiment 403, wherein said T regulatory cell expresses CTLA-4.

413. The method of embodiment 403, wherein said T regulatory cell expresses IL-10.

415. The method of embodiment 403, wherein said T regulatory cell expresses IL-35.

416. The method of embodiment 403, wherein said T regulatory cell is capable of suppressing activation of a naïve T cell.

417. The method of embodiment 416, wherein said activation of said naïve cell is proliferation of said naïve cell.

418. The method of embodiment 416, wherein said activation of said naïve cell is production of cytokines from said naïve cell.

419. The method of embodiment 418, wherein said cytokines produced by said activation of said naïve cell are selected from a group comprising of: a) IL-2; b) IL-4; c) IL-7; d) IL-9; e) IL-10; f) IL-13; g) IL-15; h) IL-17; i) IL-18; j) IL-20; k) IL-23; 1) IL-27; m) interferon gamma; and n) TNF-alpha.

420. The method of embodiment 416, wherein said activation of said naïve cell is acquisition of a memory cell phenotype.

421. The method of embodiment 416, wherein said activation of said naïve cell is acquisition of ability to induce differentiation of an immature dendritic cell.

422. The method of embodiment 421, wherein said differentiation of an immature dendritic cell is upregulation of CD40 expression.

423. The method of embodiment 421, wherein said differentiation of an immature dendritic cell is upregulation of CD80 expression.

424. The method of embodiment 421, wherein said differentiation of an immature dendritic cell is upregulation of CD86 expression.

425. The method of embodiment 421, wherein said differentiation of an immature dendritic cell is upregulation of neuropilin expression.

426. The method of embodiment 421, wherein said differentiation of an immature dendritic cell is upregulation of HLA-DR expression.

427. The method of embodiment 421, wherein said differentiation of an immature dendritic cell is upregulation of IL-12 expression.

428. The method of embodiment 421, wherein said differentiation of an immature dendritic cell is upregulation of lymph node homing chemokine receptor expression.

429. The method of embodiment 421, wherein said differentiation of an immature dendritic cell is downregulation of phagocytic activity.

430. The method of embodiment 421, wherein said differentiation of an immature dendritic cell is downregulation of IL-10 expression.

431. The method of embodiment 421, wherein said differentiation of an immature dendritic cell is upregulation of antigen presentation activity.

432. The method of embodiment 431, wherein said antigen presentation activity is ability to induce activation of a naïve T cell.

433. The method of embodiment 1 wherein said platelet rich plasma is significantly devoid of neutrophils.

434. The method of embodiment 1 wherein said platelet rich plasma possesses growth factors.

435. The method of embodiment 434, wherein said platelet rich plasma is filtered for enhancing concentration of specific growth factors.

436. The method of embodiment 435, wherein said concentration of said platelet rich plasma is performed by an affinity means.

437. The method of embodiment 436, wherein said affinity means is affinity chromatography.

438. The method of embodiment 436, wherein said affinity means is immunoadsorption.

439. The method of embodiment 436, wherein said affinity means is high pressure liquid chromatography.

440. The method of embodiment 436, wherein said affinity means is fast pressure liquid chromatography.

441. The method of embodiment 435, wherein growth factor concentrated from platelet rich plasma is Angiopoietin-like Protein 1/ANGPTL1.

442. The method of embodiment 435, wherein growth factor concentrated from platelet rich plasma is Angiopoietin-like Protein 2/ANGPTL2.

443. The method of embodiment 435, wherein growth factor concentrated from platelet rich plasma is Angiopoietin-like Protein 3/ANGPTL3.

444. The method of embodiment 435, wherein growth factor concentrated from platelet rich plasma is Angiopoietin-like Protein 4/ANGPTL4.

445. The method of embodiment 435, wherein growth factor concentrated from platelet rich plasma is Angiopoietin-like Protein 5/ANGPTL5.

446. The method of embodiment 435, wherein growth factor concentrated from platelet rich plasma is Angiopoietin-like Protein 6/ANGPTL6.

447. The method of embodiment 435, wherein growth factor concentrated from platelet rich plasma is Angiopoietin-like Protein 7/ANGPTL7.

448. The method of embodiment 435, wherein growth factor concentrated from platelet rich plasma is Angiopoietin-like Protein 8/Betatrophin.

449. The method of embodiment 435, wherein growth factor concentrated from platelet rich plasma is Angiopoietin-1.

450. The method of embodiment 435, wherein growth factor concentrated from platelet rich plasma is Angiopoietin-2.

451. The method of embodiment 435, wherein growth factor concentrated from platelet rich plasma is Angiopoietin-3.

452. The method of embodiment 435, wherein growth factor concentrated from platelet rich plasma is Angiopoietin-4.

453. The method of embodiment 435, wherein growth factor concentrated from platelet rich plasma is Amphiregulin.

454. The method of embodiment 435, wherein growth factor concentrated from platelet rich plasma is Betacellulin/BTC.

455. The method of embodiment 435, wherein growth factor concentrated from platelet rich plasma is EGF.

456. The method of embodiment 435, wherein growth factor concentrated from platelet rich plasma is EGF-L6.

457. The method of embodiment 435, wherein growth factor concentrated from platelet rich plasma is Epigen.

458. The method of embodiment 435, wherein growth factor concentrated from platelet rich plasma is Epiregulin.

459. The method of embodiment 435, wherein growth factor concentrated from platelet rich plasma is HB-EGF.

460. The method of embodiment 435, wherein growth factor concentrated from platelet rich plasma is LRIG1.

461. The method of embodiment 435, wherein growth factor concentrated from platelet rich plasma is LRIG2.

462. The method of embodiment 435, wherein growth factor concentrated from platelet rich plasma is LRIG3.

463. The method of embodiment 435, wherein growth factor concentrated from platelet rich plasma is Neuregulin-1/NRG1.

464. The method of embodiment 435, wherein growth factor concentrated from platelet rich plasma is Neuregulin-1 alpha/NRG1 alpha.

465. The method of embodiment 435, wherein growth factor concentrated from platelet rich plasma is Neuregulin-1 beta 1/NRG1 beta 1.

466. The method of embodiment 435, wherein growth factor concentrated from platelet rich plasma is Neuregulin-1 Isoform GGF2.

467. The method of embodiment 435, wherein growth factor concentrated from platelet rich plasma is Neuregulin-1 Isoform SMDF.

468. The method of embodiment 435, wherein growth factor concentrated from platelet rich plasma is Neuregulin-3/NRG3.

469. The method of embodiment 435, wherein growth factor concentrated from platelet rich plasma is TGF-alpha.

470. The method of embodiment 435, wherein growth factor concentrated from platelet rich plasma is TMEFF1/Tomoregulin-1.

471. The method of embodiment 435, wherein growth factor concentrated from platelet rich plasma is TMEFF2/Tomoregulin-2.

472. The method of embodiment 435, wherein growth factor concentrated from platelet rich plasma is EphA1.

473. The method of embodiment 435, wherein growth factor concentrated from platelet rich plasma is EphA2.

474. The method of embodiment 435, wherein growth factor concentrated from platelet rich plasma is EphA3.

475. The method of embodiment 435, wherein growth factor concentrated from platelet rich plasma is EphA4.

476. The method of embodiment 435, wherein growth factor concentrated from platelet rich plasma is EphA5.

477. The method of embodiment 435, wherein growth factor concentrated from platelet rich plasma is EphA6.

478. The method of embodiment 435, wherein growth factor concentrated from platelet rich plasma is EphA7.

479. The method of embodiment 435, wherein growth factor concentrated from platelet rich plasma is EphA8.

480. The method of embodiment 435, wherein growth factor concentrated from platelet rich plasma is EphA10.

481. The method of embodiment 435, wherein growth factor concentrated from platelet rich plasma is EphB.

482. The method of embodiment 435, wherein growth factor concentrated from platelet rich plasma is EphB1.

483. The method of embodiment 435, wherein growth factor concentrated from platelet rich plasma is EphB2.

484. The method of embodiment 435, wherein growth factor concentrated from platelet rich plasma is EphB3.

485. The method of embodiment 435, wherein growth factor concentrated from platelet rich plasma is EphB4.

486. The method of embodiment 435, wherein growth factor concentrated from platelet rich plasma is EphB6.

487. The method of embodiment 435, wherein growth factor concentrated from platelet rich plasma is Ephrin-A.

488. The method of embodiment 435, wherein growth factor concentrated from platelet rich plasma is Ephrin-A1.

489. The method of embodiment 435, wherein growth factor concentrated from platelet rich plasma is Ephrin-A2.

490. The method of embodiment 435, wherein growth factor concentrated from platelet rich plasma is Ephrin-A3.

491. The method of embodiment 435, wherein growth factor concentrated from platelet rich plasma is Ephrin-A4.

492. The method of embodiment 435, wherein growth factor concentrated from platelet rich plasma is Ephrin-A5.

493. The method of embodiment 435, wherein growth factor concentrated from platelet rich plasma is Ephrin-B.

494. The method of embodiment 435, wherein growth factor concentrated from platelet rich plasma is Ephrin-B.

495. The method of embodiment 435, wherein growth factor concentrated from platelet rich plasma is Ephrin-B1

496. The method of embodiment 435, wherein growth factor concentrated from platelet rich plasma is Ephrin-B2.

497. The method of embodiment 435, wherein growth factor concentrated from platelet rich plasma is Ephrin-B3.

498. The method of embodiment 435, wherein growth factor concentrated from platelet rich plasma is FGF-1.

499. The method of embodiment 435, wherein growth factor concentrated from platelet rich plasma is FGF-2.

500. The method of embodiment 435, wherein growth factor concentrated from platelet rich plasma is FGF-3.

501. The method of embodiment 435, wherein growth factor concentrated from platelet rich plasma is FGF-4.

502. The method of embodiment 435, wherein growth factor concentrated from platelet rich plasma is FGF-5.

503. The method of embodiment 435, wherein growth factor concentrated from platelet rich plasma is FGF-6

504. The method of embodiment 435, wherein growth factor concentrated from platelet rich plasma is KGF.

505. The method of embodiment 435, wherein growth factor concentrated from platelet rich plasma is FGF-8.

506. The method of embodiment 435, wherein growth factor concentrated from platelet rich plasma is FGF-9.

507. The method of embodiment 435, wherein growth factor concentrated from platelet rich plasma is FGF-10.

508. The method of embodiment 435, wherein growth factor concentrated from platelet rich plasma is FGF-11.

509. The method of embodiment 435, wherein growth factor concentrated from platelet rich plasma is FGF-12.

510. The method of embodiment 435, wherein growth factor concentrated from platelet rich plasma is FGF-13.

511. The method of embodiment 435, wherein growth factor concentrated from platelet rich plasma is FGF-15.

512. The method of embodiment 435, wherein growth factor concentrated from platelet rich plasma is FGF-16.

513. The method of embodiment 435, wherein growth factor concentrated from platelet rich plasma is FGF-17.

514. The method of embodiment 435, wherein growth factor concentrated from platelet rich plasma is FGF-18.

515. The method of embodiment 435, wherein growth factor concentrated from platelet rich plasma is FGF-19.

516. The method of embodiment 435, wherein growth factor concentrated from platelet rich plasma is FGF-20.

517. The method of embodiment 435, wherein growth factor concentrated from platelet rich plasma is FGF-21.

518. The method of embodiment 435, wherein growth factor concentrated from platelet rich plasma is FGF-22.

519. The method of embodiment 435, wherein growth factor concentrated from platelet rich plasma is FGF-23.

520. The method of embodiment 435, wherein growth factor concentrated from platelet rich plasma is alpha 2-Macroglobulin.

521. The method of embodiment 435, wherein growth factor concentrated from platelet rich plasma is alpha 2-Macroglobulin-like 1/A2ML1.

522. The method of embodiment 435, wherein growth factor concentrated from platelet rich plasma is CNPY2.

523. The method of embodiment 435, wherein growth factor concentrated from platelet rich plasma is FGF-BP.

524. The method of embodiment 435, wherein growth factor concentrated from platelet rich plasma is FGFBP2.

525. The method of embodiment 435, wherein growth factor concentrated from platelet rich plasma is FGFBP3.

526. The method of embodiment 435, wherein growth factor concentrated from platelet rich plasma is FRS2.

527. The method of embodiment 435, wherein growth factor concentrated from platelet rich plasma is Klotho.

528. The method of embodiment 435, wherein growth factor concentrated from platelet rich plasma is Klotho beta.

529. The method of embodiment 435, wherein growth factor concentrated from platelet rich plasma is LRIT3.

530. The method of embodiment 435, wherein growth factor concentrated from platelet rich plasma is Pentraxin 3/TSG-14.

531. The method of embodiment 435, wherein growth factor concentrated from platelet rich plasma is Shisa-4.

532. The method of embodiment 435, wherein growth factor concentrated from platelet rich plasma is SPRY1.

533. The method of embodiment 435, wherein growth factor concentrated from platelet rich plasma is SPRY2.

534. The method of embodiment 435, wherein growth factor concentrated from platelet rich plasma is SPRY3.

535. The method of embodiment 435, wherein growth factor concentrated from platelet rich plasma is Desert Hedgehog/Dhh.

536. The method of embodiment 435, wherein growth factor concentrated from platelet rich plasma is

Indian Hedgehog/Ihh.

537. The method of embodiment 435, wherein growth factor concentrated from platelet rich plasma is Sonic Hedgehog/Shh.

538. The method of embodiment 435, wherein growth factor concentrated from platelet rich plasma is Hedgehog Related Molecules and Regulators.

539. The method of embodiment 435, wherein growth factor concentrated from platelet rich plasma is beta-TrCP1/BTRC.

540. The method of embodiment 435, wherein growth factor concentrated from platelet rich plasma is BOC.

541. The method of embodiment 435, wherein growth factor concentrated from platelet rich plasma is C2CD3.

542. The method of embodiment 435, wherein growth factor concentrated from platelet rich plasma is CDO.

543. The method of embodiment 435, wherein growth factor concentrated from platelet rich plasma is DISP1.

544. The method of embodiment 435, wherein growth factor concentrated from platelet rich plasma is DISP2.

545. The method of embodiment 435, wherein growth factor concentrated from platelet rich plasma is Gas 1.

546. The method of embodiment 435, wherein growth factor concentrated from platelet rich plasma is GLI-1.

547. The method of embodiment 435, wherein growth factor concentrated from platelet rich plasma is GLI-2.

548. The method of embodiment 435, wherein growth factor concentrated from platelet rich plasma is GLI-3.

549. The method of embodiment 435, wherein growth factor concentrated from platelet rich plasma is Glypican 3.

550. The method of embodiment 435, wherein growth factor concentrated from platelet rich plasma is Glypican 4.

551. The method of embodiment 435, wherein growth factor concentrated from platelet rich plasma is GPR161.

552. The method of embodiment 435, wherein growth factor concentrated from platelet rich plasma is GSK-3 alpha/beta.

553. The method of embodiment 435, wherein growth factor concentrated from platelet rich plasma is GSK-3 alpha.

554. The method of embodiment 435, wherein growth factor concentrated from platelet rich plasma is GSK-3 beta.

555. The method of embodiment 435, wherein growth factor concentrated from platelet rich plasma is Hedgehog Signaling Activators.

556. The method of embodiment 435, wherein growth factor concentrated from platelet rich plasma is Hedgehog Signaling Inhibitors.

557. The method of embodiment 435, wherein growth factor concentrated from platelet rich plasma is HHAT.

558. The method of embodiment 435, wherein growth factor concentrated from platelet rich plasma is HHIPL1.

559. The method of embodiment 435, wherein growth factor concentrated from platelet rich plasma is HHIPL2.

560. The method of embodiment 435, wherein growth factor concentrated from platelet rich plasma is Hip.

561. The method of embodiment 435, wherein growth factor concentrated from platelet rich plasma is LIN-41.

562. The method of embodiment 435, wherein growth factor concentrated from platelet rich plasma is Patched 1/PTCH.

563. The method of embodiment 435, wherein growth factor concentrated from platelet rich plasma is Patched 2/PTCH2.

564. The method of embodiment 435, wherein growth factor concentrated from platelet rich plasma is SCUBE3.

565. The method of embodiment 435, wherein growth factor concentrated from platelet rich plasma is smoothened

566. The method of embodiment 435, wherein growth factor concentrated from platelet rich plasma is TCTN1.

567. The method of embodiment 435, wherein growth factor concentrated from platelet rich plasma is HTRA4.

568. The method of embodiment 435, wherein growth factor concentrated from platelet rich plasma is IGFBP-1.

569. The method of embodiment 435, wherein growth factor concentrated from platelet rich plasma is IGFBP-2.

570. The method of embodiment 435, wherein growth factor concentrated from platelet rich plasma is IGFBP-3.

571. The method of embodiment 435, wherein growth factor concentrated from platelet rich plasma is IGFBP-4.

572. The method of embodiment 435, wherein growth factor concentrated from platelet rich plasma is IGFBP-5.

573. The method of embodiment 435, wherein growth factor concentrated from platelet rich plasma is IGFBP-6.

574. The method of embodiment 435, wherein growth factor concentrated from platelet rich plasma is IGFBP-L1

575. The method of embodiment 435, wherein growth factor concentrated from platelet rich plasma is IGFBP-rp1/IGFBP-7

576. The method of embodiment 435, wherein growth factor concentrated from platelet rich plasma is IGFBP-rP10

577. The method of embodiment 435, wherein growth factor concentrated from platelet rich plasma is IGF-I/IGF-1.

578. The method of embodiment 435, wherein growth factor concentrated from platelet rich plasma is IGF-II/IGF2

579. The method of embodiment 435, wherein growth factor concentrated from platelet rich plasma is IGFL-3

580. The method of embodiment 435, wherein growth factor concentrated from platelet rich plasma is CILP-1

581. The method of embodiment 435, wherein growth factor concentrated from platelet rich plasma is CTGF/CCN2.

582. The method of embodiment 435, wherein growth factor concentrated from platelet rich plasma is Cyr61/CCN1

583. The method of embodiment 435, wherein growth factor concentrated from platelet rich plasma is Endocan/ESM-1.

584. The method of embodiment 435, wherein growth factor concentrated from platelet rich plasma is IGFALS/ALS.

585. The method of embodiment 435, wherein growth factor concentrated from platelet rich plasma is IMP2.

586. The method of embodiment 435, wherein growth factor concentrated from platelet rich plasma is NOV/CCN3.

587. The method of embodiment 435, wherein growth factor concentrated from platelet rich plasma is TMEM219.

588. The method of embodiment 435, wherein growth factor concentrated from platelet rich plasma is WISP-1/CCN4.

589. The method of embodiment 435, wherein growth factor concentrated from platelet rich plasma is WISP3

590. The method of embodiment 435, wherein growth factor concentrated from platelet rich plasma is PDGF-A.

591. The method of embodiment 435, wherein growth factor concentrated from platelet rich plasma is PDGF-AA.

592. The method of embodiment 435, wherein growth factor concentrated from platelet rich plasma is PDGF-AB.

593. The method of embodiment 435, wherein growth factor concentrated from platelet rich plasma is PDGF-AB/BB.

594. The method of embodiment 435, wherein growth factor concentrated from platelet rich plasma is PDGF-B.

595. The method of embodiment 435, wherein growth factor concentrated from platelet rich plasma is PDGF-BB.

596. The method of embodiment 435, wherein growth factor concentrated from platelet rich plasma is PDGF-C.

597. The method of embodiment 435, wherein growth factor concentrated from platelet rich plasma is PDGF-CC.

598. The method of embodiment 435, wherein growth factor concentrated from platelet rich plasma is PDGF-D.

599. The method of embodiment 435, wherein growth factor concentrated from platelet rich plasma is PDGF-DD.

600. The method of embodiment 435, wherein growth factor concentrated from platelet rich plasma is HGF-1.

601. The method of embodiment 435, wherein growth factor concentrated from platelet rich plasma is Decorin.

602. The method of embodiment 435, wherein growth factor concentrated from platelet rich plasma is Glypican 1.

603. The method of embodiment 435, wherein growth factor concentrated from platelet rich plasma is Glypican 2.

604. The method of embodiment 435, wherein growth factor concentrated from platelet rich plasma is Glypican 3.

605. The method of embodiment 435, wherein growth factor concentrated from platelet rich plasma is Glypican 4.

606. The method of embodiment 435, wherein growth factor concentrated from platelet rich plasma is Glypican 5.

607. The method of embodiment 435, wherein growth factor concentrated from platelet rich plasma is Glypican 6.

608. The method of embodiment 435, wherein growth factor concentrated from platelet rich plasma is aggrecan.

609. The method of embodiment 435, wherein growth factor concentrated from platelet rich plasma is Brevican.

610. The method of embodiment 435, wherein growth factor concentrated from platelet rich plasma is Neurocan.

611. The method of embodiment 435, wherein growth factor concentrated from platelet rich plasma is versican.

612. The method of embodiment 435, wherein growth factor concentrated from platelet rich plasma is Syndecan-1/CD138.

613. The method of embodiment 435, wherein growth factor concentrated from platelet rich plasma is Syndecan-2/CD362.

614. The method of embodiment 435, wherein growth factor concentrated from platelet rich plasma is Syndecan-3.

615. The method of embodiment 435, wherein growth factor concentrated from platelet rich plasma is Syndecan-4

616. The method of embodiment 435, wherein growth factor concentrated from platelet rich plasma is Testican 1/SPOCK1.

617. The method of embodiment 435, wherein growth factor concentrated from platelet rich plasma is Testican 2/SPOCK2.

618. The method of embodiment 435, wherein growth factor concentrated from platelet rich plasma is Testican 3/SPOCK3.

619. The method of embodiment 435, wherein growth factor concentrated from platelet rich plasma is Agrin.

620. The method of embodiment 435, wherein growth factor concentrated from platelet rich plasma is alpha-Sarcoglycan.

621. The method of embodiment 435, wherein growth factor concentrated from platelet rich plasma is beta-Sarcoglycan.

622. The method of embodiment 435, wherein growth factor concentrated from platelet rich plasma is Biglycan.

623. The method of embodiment 435, wherein growth factor concentrated from platelet rich plasma is Bikunin.

624. The method of embodiment 435, wherein growth factor concentrated from platelet rich plasma is CHADL.

625. The method of embodiment 435, wherein growth factor concentrated from platelet rich plasma is Chondroadherin.

626. The method of embodiment 435, wherein growth factor concentrated from platelet rich plasma is Cytokeratin 18.

627. The method of embodiment 435, wherein growth factor concentrated from platelet rich plasma is DSPG3.

628. The method of embodiment 435, wherein growth factor concentrated from platelet rich plasma is Dystroglycan.

629. The method of embodiment 435, wherein growth factor concentrated from platelet rich plasma is Endocan/ESM-1.

630. The method of embodiment 435, wherein growth factor concentrated from platelet rich plasma is Endoglycan/PODXL2.

631. The method of embodiment 435, wherein growth factor concentrated from platelet rich plasma is Endorepellin/Perlecan.

632. The method of embodiment 435, wherein growth factor concentrated from platelet rich plasma is epsilon-Sarcoglycan.

633. The method of embodiment 435, wherein growth factor concentrated from platelet rich plasma is Fibromodulin/FMOD.

634. The method of embodiment 435, wherein growth factor concentrated from platelet rich plasma is Keratocan.

635. The method of embodiment 435, wherein growth factor concentrated from platelet rich plasma is Lumican.

636. The method of embodiment 435, wherein growth factor concentrated from platelet rich plasma is MBP-1.

637. The method of embodiment 435, wherein growth factor concentrated from platelet rich plasma is Mimecan.

638. The method of embodiment 435, wherein growth factor concentrated from platelet rich plasma is Neuroglycan C/CSPG5.

639. The method of embodiment 435, wherein growth factor concentrated from platelet rich plasma is NG2/MCSP.

640. The method of embodiment 435, wherein growth factor concentrated from platelet rich plasma is NYX.

641. The method of embodiment 435, wherein growth factor concentrated from platelet rich plasma is Opticin.

642. The method of embodiment 435, wherein growth factor concentrated from platelet rich plasma is Osteoadherin/OSAD.

643. The method of embodiment 435, wherein growth factor concentrated from platelet rich plasma is Podocan.

644. The method of embodiment 435, wherein growth factor concentrated from platelet rich plasma is PRELP.

645. The method of embodiment 435, wherein growth factor concentrated from platelet rich plasma is PRG3/MBP2.

646. The method of embodiment 435, wherein growth factor concentrated from platelet rich plasma is delta-Sarcoglycan.

647. The method of embodiment 435, wherein growth factor concentrated from platelet rich plasma is TGF-beta RIII.

648. The method of embodiment 435, wherein growth factor concentrated from platelet rich plasma is PlGF-2.

649. The method of embodiment 435, wherein growth factor concentrated from platelet rich plasma is PlGF-3.

650. The method of embodiment 435, wherein growth factor concentrated from platelet rich plasma is PlGF-4.

651. The method of embodiment 435, wherein growth factor concentrated from platelet rich plasma is VEGF/PlGF Heterodimer.

652. The method of embodiment 435, wherein growth factor concentrated from platelet rich plasma is VEGF-B.

653. The method of embodiment 435, wherein growth factor concentrated from platelet rich plasma is VEGF-C.

654. The method of embodiment 435, wherein growth factor concentrated from platelet rich plasma is VEGF-D

654. The method of embodiment 435, wherein growth factor concentrated from platelet rich plasma is Chondromodulin-1.

655. The method of embodiment 435, wherein growth factor concentrated from platelet rich plasma is CTGF/CCN2.

656. The method of embodiment 435, wherein growth factor concentrated from platelet rich plasma is Cyr61/CCN1.

657. The method of embodiment 435, wherein growth factor concentrated from platelet rich plasma is EG-VEGF/PK1.

658. The method of embodiment 435, wherein growth factor concentrated from platelet rich plasma is Fibulin 1.

659. The method of embodiment 435, wherein growth factor concentrated from platelet rich plasma is Fibulin 2.

660. The method of embodiment 435, wherein growth factor concentrated from platelet rich plasma is Fibulin 3.

661. The method of embodiment 435, wherein growth factor concentrated from platelet rich plasma is Fibulin 5/DANCE.

662. The method of embodiment 435, wherein growth factor concentrated from platelet rich plasma is Fibulin 7.

663. The method of embodiment 435, wherein growth factor concentrated from platelet rich plasma is HDGF

664. The method of embodiment 435, wherein growth factor concentrated from platelet rich plasma is Hepassocin/FGL1.

665. The method of embodiment 435, wherein growth factor concentrated from platelet rich plasma is LECT2.

666. The method of embodiment 435, wherein growth factor concentrated from platelet rich plasma is LEDGF.

667. The method of embodiment 435, wherein growth factor concentrated from platelet rich plasma is LRRN1/NLRR-1.

668. The method of embodiment 435, wherein growth factor concentrated from platelet rich plasma is LYAR.

669. The method of embodiment 435, wherein growth factor concentrated from platelet rich plasma is beta-NGF.

670. The method of embodiment 435, wherein growth factor concentrated from platelet rich plasma is Norrin.

671. The method of embodiment 435, wherein growth factor concentrated from platelet rich plasma is NOV/CCN3.

672. The method of embodiment 435, wherein growth factor concentrated from platelet rich plasma is Oncomodulin

673. The method of embodiment 435, wherein growth factor concentrated from platelet rich plasma is Osteocrin.

674. The method of embodiment 435, wherein growth factor concentrated from platelet rich plasma is PD-ECGF.

675. The method of embodiment 435, wherein growth factor concentrated from platelet rich plasma is Progranulin/PGRN.

676. The method of embodiment 435, wherein growth factor concentrated from platelet rich plasma is S100A13.

677. The method of embodiment 435, wherein growth factor concentrated from platelet rich plasma is SF20/MYDGF.

678. The method of embodiment 435, wherein growth factor concentrated from platelet rich plasma is SLURP1.

679. The method of embodiment 435, wherein growth factor concentrated from platelet rich plasma is SLURP2.

680. The method of embodiment 435, wherein growth factor concentrated from platelet rich plasma is Thrombopoietin/Tpo.

681. The method of embodiment 435, wherein growth factor concentrated from platelet rich plasma is WISP-1/CCN4

682. The method of embodiment 435, wherein growth factor concentrated from platelet rich plasma is WISP3.

683. The method of embodiment 1, wherein said anti-inflammatory/regenerative adjuvant is Calagualine (fern derivative).

684. The method of embodiment 1, wherein said anti-inflammatory/regenerative adjuvant is Conophylline (Ervatamia microphylla).

685. The method of embodiment 1, wherein said anti-inflammatory/regenerative adjuvant is Evodiamine (Evodiae fructus component).

686. The method of embodiment 1, wherein said anti-inflammatory/regenerative adjuvant is Geldanamycin.

687. The method of embodiment 1, wherein said anti-inflammatory/regenerative adjuvant is Perrilyl alcohol.

688. The method of embodiment 1, wherein said anti-inflammatory/regenerative adjuvant is pterostilbene.

689. The method of embodiment 1, wherein said anti-inflammatory/regenerative adjuvant is Protein-bound polysaccharide from basidiomycetes.

690. The method of embodiment 1, wherein said anti-inflammatory/regenerative adjuvant is Rocaglamides (Aglaia derivatives).

691. The method of embodiment 1, wherein said anti-inflammatory/regenerative adjuvant is 15-deoxy-prostaglandin J(2).

692. The method of embodiment 1, wherein said anti-inflammatory/regenerative adjuvant is lithium.

693. The method of embodiment 1, wherein said anti-inflammatory/regenerative adjuvant is Anandamide.

694. The method of embodiment 1, wherein said anti-inflammatory/regenerative adjuvant is Artemisia vestita.

695. The method of embodiment 1, wherein said anti-inflammatory/regenerative adjuvant is Cobrotoxin.

696. The method of embodiment 1, wherein said anti-inflammatory/regenerative adjuvant is Dehydroascorbic acid (Vitamin C).

697. The method of embodiment 1, wherein said anti-inflammatory/regenerative adjuvant is Herbimycin A.

698. The method of embodiment 1, wherein said anti-inflammatory/regenerative adjuvant is Isorhapontigenin.

699. The method of embodiment 1, wherein said anti-inflammatory/regenerative adjuvant is Manumycin A.

700. The method of embodiment 1, wherein said anti-inflammatory/regenerative adjuvant is Pomegranate fruit extract.

701. The method of embodiment 1, wherein said anti-inflammatory/regenerative adjuvant is selected from a group comprising of: Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2C1), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic)

### BRIEF DESCRIPTION OF THE DRAWINGS

FIG. 1 is a bar graph showing the reduction of brain microglial activation by PRP and Pterostilbene.

FIG. 2 is a bar graph showing the reduction of brain microglial activation by PRP and Oxytocin.

### DESCRIPTION OF THE INVENTION

As will be apparent to those of skill in the art upon reading this disclosure, each of the individual aspects described and illustrated herein has discrete components and features which may be readily separated from or combined with the features of any of the other several aspects without departing from the scope or spirit of the present disclosure. Any recited method can be carried out in the order of events recited or in any other order that is logically possible.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a neuropsychiatric disorder (including, but not limited to, PTSD, or a symptom thereof. Others are described elsewhere herein). The effect can be therapeutic in terms of a partial or complete cure of a disease, condition, symptom or adverse effect attributed to the disease, disorder, or condition. The term “treatment” as used herein covers any treatment of a neuropsychiatric disorder (including, but not limited to, PTSD or a symptom thereof), in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic (preventative) treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

Neuropsychiatric disorders are generally diseases, conditions, and disorders of affect, cognition, and/or behavior that can arise from an overt disorder in cerebral function or from indirect effects of extracerebral diseases and disorders. Neuropsychiatric disorders are a significant burden on society and can impair the health of those affected, as well as their ability to learn, work, and/or emotionally cope. They also can burden those not afflicted in that those affected often must rely on caregivers or other forms of assistance due to their inability to fully engage and function in normal work and life activities. Non-limiting examples of neuropsychiatric disorders include addiction, developmental conditions (e.g. attention deficit hyperactivity disorder (ADHD), autism, fetal alcohol syndrome, and tic disorders), eating disorders, degenerative disease (e.g. dementia, Parkinson's disease, and Alzheimer's disease), mood/affect disorders (e.g. bipolar disorder, depressions, and mania), neurotic disorders (e.g. obsessive compulsive disorder, trichotillomania, and anxiety disorders (including post-traumatic stress disorder (PTSD)), psychosis (e.g. schizophrenia), and sleep disorders (e.g. sleep apnea, narcolepsy, insomnia, and parasomnia).

“Substance addiction”, substance abuse, substance dependence, or substance use disorder: Substance addiction, substance abuse, substance dependence, and substance use disorder are used interchangeably herein. A substance refers to any legal or illegal drug, medication, or toxin. The Diagnostic and Statistical Manual of Mental Disorders (DSM-IV) defines a Substance Use Disorder as a maladaptive pattern of substance use leading to clinically significant impairment or distress. The DSM-IV currently differentiates between two separate Substance Use Disorders (Substance Abuse and Substance Dependence); however, the DSM-V is scheduled for release in 2013 and the proposed draft revision eliminates this distinction and instead combines criteria into a single disorder (Substance Use Disorder). Physiological dependence occurs only in a subset of individuals with a Substance Use Disorder and includes tolerance to the substance and the appearance of characteristic withdrawal symptoms (for example, increased heart rate and/or blood pressure, sweating, tremors, confusion, convulsions, and visual hallucinations) when the substance is suddenly discontinued. For individuals who meet DSM-IV criteria for Substance Dependence (or in the future, for those meeting DSM-V criteria for Substance Use Disorder), the specifiers “With Physiological Dependence” or “Without Physiological Dependence” are used to indicate the presence or absence of tolerance and/or withdrawal symptoms. Additionally, “substance dependence” as used herein, refers to a pattern of substance use, leading to clinically significant impairment or distress as manifested by at least three selected from the following group, occurring at any time within a period of time: (1) tolerance as defined by either (a) a need for substantially increased amounts of the substance to achieve the desired effect; or (b) substantially diminished effect with continued use of the same amount of the substance; (2) withdrawal, as demonstrated by either (a) the characteristic withdrawal syndrome for the specific substance; or (b) the same, or a closely related substance is taken to relieve or avoid withdrawal symptoms; (3) the substance is often taken in larger amounts or over a longer period then was intended; (4) there is a persistent desire or unsuccessful efforts to cut down or control substance use; (5) a great deal of time is spent in activities to obtain the substance, use the substance, or recover from its effects; (6) important social, occupational or recreational activities are given up or reduced because of substance use; and (7) the substance use is continued despite knowledge of having a persistent or recurrent physical or psychological problem that is likely to have been caused or exacerbated by the substance. Substance dependence can be with physiological dependence; that is evidence of tolerance or withdrawal is present, or without physiological dependence, where no evidence of tolerance or withdrawal is present.[6]

There are several reasons to believe that neuroinflammation is associated with addiction. For example, opioids have been demonstrated to activate and/or prime glial cells in cases of activation [40-47]. Additionally, addiction to pain killers is in many times associated with pain, and it is believed that uncontrolled activation of microglial cells under neuropathic pain conditions induces the release of proinflammatory cytokines such as (interleukin—IL-1beta, IL-6, tumor necrosis factor—TNF-alpha), complement components (C1q, C3, C4, C5, C5a). These inflammatory mediators are believed, in part, to facilitate pain transmission. Furthermore, microglia activation can lead to altered activity of opioid systems and neuropathic pain is characterized by resistance to morphine. Pharmacological attenuation of glial activation represents a novel approach for controlling neuropathic pain. It has been found that propentofylline, pentoxifylline, fluorocitrate and minocycline decrease microglial activation and inhibit proinflammatory cytokines, thereby suppressing the development of neuropathic pain [48, 49]. In some embodiments of the invention, the use of opioids for decreasing neuropathic pain is reduced because administration of agents that inhibit glial cell activation is give together with probiotic, and/or probiotic enzyme complex in order to inhibit pain, reduce tolerance, and reduce need for addiction.

Through the practice of the invention, various addictive attributes can be targeted, as well as molecules associated with induction of addiction. In one embodiment, the invention teaches that pain mediators that may be targeted together with administration of probiotic, and/or probiotic enzyme mixture, as well as pterostilbene, and/or oxytocin and/or platelet rich plasma is interleukin 6. It is known that spinal IL-6 levels correlated directly with the mechanical allodynia intensity following nerve injury. One study sought to determine whether it is possible to attenuate mechanical allodynia and/or alter spinal glial activation resulting from peripheral nerve injury by specific manipulation of IL-6 with neutralizing antibodies or by global immune modulation utilizing immunogamma-globulin (IgG). Effects of peripheral administration of normal goat IgG and intrathecal (i.t.) administration of IL-6 neutralizing antibody, normal goat or normal rat IgG on mechanical allodynia associated with L5 spinal nerve transection were compared. Spinal glial activation was assessed at day 10 post surgery by immunohistochemistry. Low dose (0.01-0.001 microg) goat anti-rat IL-6 i.t. administration (P=0.025) significantly decreased allodynia and trended towards significance at the higher dose (0.08 microg to 0.008 microg, P=0.062). Low doses (0.01-0.001 microg) i.t. normal goat and rat IgG significantly attenuated mechanical allodynia, but not at higher doses (0.08-0.008 microg; P=0.001 for both goat and rat IgG). Peripherally administered normal goat IgG (30 or 100 mg/kg) did not attenuate mechanical allodynia. Thus in one embodiment of the invention, neutralization of IL-6 may be performed together with administration of probiotic, and/or probiotic/enzyme mixture, for the treatment of pain, and reduction of addiction associated with opioids [50]. The role of IL-6 in addiction is well known and one of skill in the art is referred to the following publications which are incorporated by reference [51-58].

In some studies a direct correlation has been shown between IL-6 levels and alcohol addiction. For example, Heberlein et al investigated the serum levels of IL-6 and TNF-α in 30 male alcohol-dependent patients during withdrawal (day 1, 7, and 14) and compared them with the levels obtained from 18 healthy male controls. IL-6 (day 1: T=2,593, p=0.013; day 7: T=2,315, p=0.037; day 14: T=1,650, p=0.112) serum levels were significantly increased at the beginning of alcohol withdrawal. TNF-α (T=3,202, p=0.03) serum levels were significantly elevated in the patients' group during the whole period of withdrawal. IL-6 serum levels decreased significantly during withdrawal (F=16.507, p<0.001), whereas TNF-α levels did not change significantly (day 1-14). IL-6 serum levels were directly associated with alcohol consumption (r=0.392, p=0.047) on day 1. Moreover, the IL-6 serum levels were associated with alcohol craving (PACS total score day 1: r=−0.417, p=0.022, the score of the obsessive subscale of the OCDS on day 14 [r=−0.549, p=0.022]), depression (r=−0.507, p=0.005), and trait anxiety (r=−0.674, p<0.001) on day 1. The authors found an association with the duration of active drinking following the last period of abstinence and the TNF-α serum levels (day 1:r=0.354, p=0.009; day 7: r=0.323, p=0.022; day 14: r=0.303, p=0.034) as well as an association with the severity of alcohol dependence measured by the SESA scale (r=0.454, p=0.015) [59].

In one embodiment of the invention, the PRP and/or oxytocin and/or pterostilbene mixture is administered in order to enhance neurogenesis in patients who have suffered from brain damage as a result of opioid and/or other addictions. The study of adult neurogenesis has been previously described [60-63], and suppression of neurogenesis in alcohol, opioid, cocaine and methamphetamine addiction has been previously reported [64-74]. One of the aims of the current disclosure is to induce, and/or repair brain tissue that has been damage.

In one embodiment of the invention, stem cells are utilized to overcome drug induced neurotoxicity [75], and said PRP and/or oxytocin and/or pterostilbene mixture is utilized to enhance efficacy of stem cells. Enhancement of efficacy comprises stimulation of growth factor production, inhibition of inflammation and triggering of mitogenesis of stem cells and existing endogenous regenerative cells.

In another embodiment of the invention, the PRP and/or oxytocin and/or pterostilbene mixture is administered together with growth factors and/or hormones to stimulate regeneration of injured brain cells and decrease abnormalities that have been associated with suppression of neural pathways by addiction. Example include administration of growth hormone [76], agents which block adrenal hormones [77, 78], enhancement of serotonin [79-81], administration of FGF-2 [82, 83], antidepressants such as tranylcypromine, reboxetine, fluoxetine, haloperidol, tranylcypromine, reboxetine, fluoxetine, and haloperidol [84], electroconvulsive therapy [85], lithium [86], insulin like growth factor [87], inhibition of IL-6 [88], heparin binding epidermal growth factor like growth factor [89], VEGF [90], DHEA [91], BDNF [92], NMDA receptor antagonists [93], PGE2 [94], prolactin [95], the Chronic AMPA receptor potentiator (LY451646) [96], PACAP [97], lithium [98], transcranial magnetic field stimulation [99], olanzapine or fluoxetine [100],

In some embodiments, the PRP and/or oxytocin and/or pterostilbene mixture is utilized together with reducers of oxidative stress to decrease neuronal cell death. It has been shown that adult neurogenesis within the dentate gyrus of the hippocampus is selectively impaired in a rat model of alcoholism, and that it can be completely prevented by the antioxidant ebselen. Rats fed for 6 weeks with a liquid diet containing moderate doses of ethanol had a 66.3% decrease in the number of new neurons and a 227-279% increase in cell death in the dentate gyrus as compared with paired controls. Neurogenesis within the olfactory bulb was not affected by alcohol. These studies indicate that alcohol abuse, even for a short duration, results in the death of newly formed neurons within the adult brain and that the underlying mechanism is related to oxidative or nitrosative stress [101].

In another embodiment of the invention, the PRP and/or oxytocin and/or pterostilbene mixture is administered together with transcranial magnetic stimulation

In some embodiments of the invention, patients at risk for opioid addictions/or neuropsychiatric conditions are identified by on oxidative stress. Oxidative damage in the tissues and cells of an individual may be the result of reactive oxygen species (ROS), such as peroxides and oxygen radicals. Some level of ROS is normal in an organism and certain ROS species take part in normal biochemical pathways. However, excessive ROS levels cause oxidation of certain biomolecules in an individual, and the oxidation products, or derivatives therefrom, may appear in bodily fluids such as blood or urine. ROS can be produced from fungal or viral infection, ageing, UV radiation, pollution, excessive alcohol consumption, and cigarette smoking among other diseases. ROS can further cause age-related macular degeneration and cataracts. Of primary interest are the oxidation products of certain fatty acids and DNA, as the appearance of the oxidation products from fatty acids or DNA can be indicative of excessive ROS and the existence of oxidative damage at the cellular level. Oxidation of fatty acids in an organism is often referred to as “lipid peroxidation.” Further, ROS also includes the reactive nitrogen species (RNS), which includes nitric oxide radical NO and ONO.sub.2-. These reactive species cause “nitrative stress,” with RNS reaction products including such molecules as 3-nitrotyrosine. Since the RNS species are in effect ROS species, nitrative stress is normally lumped together with oxidative stress when referring to oxidative damage in individuals. In lipid peroxidation, it is the unsaturated fats that are most prone to oxidation, particularly arachidonic acid and linoleic acid with their polyunsaturated carbon chains. For example, oxidation of arachidonic acid and linoleic acid produces malondialdehyde (MDA) and 4-hydroxynonenal (4-HNE), amongst other products, which are secreted in the urine. MDA and 4-HNE can be measured in a urine sample as oxidative stress biomarkers. These biomarkers can be used to assess risk of opioid addiction as well as to predict response to therapies described in the current patent. An oxidative stress assay may comprise a specific malondialdehyde (MDA) or 4-hydroxyonenal (4HNE) method to quantify lipid peroxidation and/or a thiobarbituric acid reactive substances (TBARS) method to measure a broader range of substances oxidized to aldehydes and ketones due to the actions of free radicals. A ferrous reaction reagent suitable for use in assaying oxidative stress comprises 2-deoxyglucose, TBA, EDTA, and ferrous sulfate. Oxidized derivatives of amino acids in proteins are also biomarkers of oxidative stress. In principle, an oxidative stress biomarker can be any amino acid that has undergone oxidation or some other modification. For example, dityrosine and 3-nitrotyrosine are oxidative stress biomarkers produced by the reaction of tyrosine with peroxynitrite, or chloro-tyrosine, which is produced by the action of myeloperoxidase and is an inflammatory biomarker. Urinary 3-nitrotyrosine excretion is a urinary biomarker that reflects excessive ROS in an individual, such as ONO.sub.2-. 3-Nitrotyrosine is the major product of tyrosine oxidation, although it is not clear if tyrosine is oxidized when in free form or when part of a polypeptide. See, for example, Radi, R., “Nitric oxide, oxidants, and protein tyrosine nitration,” Proc. Natl, Acad. Sci., 101, 4003-4008 (2004). Further, oxidized sulfur- or selenium-containing amino acids (collectively referred to as “SSAA”) are oxidative stress biomarkers. Oxidized SSAA are amino acids in which the sulfur or selenium moiety has been oxidized to a higher oxidation state. Oxidized SSAA include, but are not limited to, cysteine, cystine, methionine, selenomethionine, selenocystine and selenocysteine in their various possible oxidation states. In general, high levels of any one of these biomarkers indicate that oxidative stress is occurring in an individual. Low levels of these biomarkers indicate a healthy individualAdditional oxidation and nitration products of lipids, proteins and DNA that find use as oxidative stress biomarkers include isoprostanes, 8-hydroxyguanosine and 8-hydroxy-2′deoxyguanosine. Oxidative damage to DNA can be evidenced by oxidation products of the most susceptible base, guanosine. The oxidation products that can be found at elevated levels in urine when excessive ROS are present include 8-hydroxyguanosine and 8-hydroxy-2′-guanosine. These substances have been shown to be useful biomarkers of oxidative stress. See, for example, Shigenaga, M. K., et al., “Urinary 8-hydroxy-2′deoxyguanosine as a biological marker of in vivo oxidative DNA damage,” Proc. Natl. Acad. Sci., 86, 9697-9701 (1989). Isoprostanes found in urine primarily consist of 8-iso-prostaglandin F.sub.2.alpha., referred to more simply herein as F2-isoprostane, or F2-isoP. F2-isoPs are chemically stable prostaglandin-like isomers, generated by the reaction of polyunsaturated fatty acids and ROS, and have been shown to be useful biomarkers for oxidative stress in an individual. See, for example, Cracowski, J.-L., et al., “Isoprostanes as a biomarker of lipid peroxidation in humans: physiology, pharmacology and clinical implications,” Trends Pharmacol. Sci., 23, 360-366 (2002)). Glutathione (GSH) is a tripeptide molecule that acts as an antioxidant, reducing various ROS species to become oxidized to the disulfide, GSSG. Since both the oxidized (GSH) and reduced (GSSG) species exist naturally, what is more important for health assessment is the ratio of GSH/GSSG. This ratio is about 30-100 in cytosol of cells, and about 3-10 in serum. The ratio decreases in the presence of oxidative stress. That is, there is an abnormally low level of GSH, and abnormally high level of GSSG, or both, causing the GSH/GSSG ratio to be lower than normal. See, in general, Frijhoff, J., et al., “Clinical relevance of biomarkers of oxidative stress,” Antioxid. Redox Signal., 23(14), 1144-70 (2015). Uric acid is a degradation product of purine, and is indicative of an inflammatory factor that increases oxidative stress and promotes activation of the renin angiotensin aldosterone system. Thus uric acid is a useful urinary biomarker indicative of oxidative stress and overall health. N-hexanoyl lysise (HEL), or more simply, hexanoyl-lysine adduct, is another lipid peroxidation biomarker. It is the product of omega-6 polyunsaturated fatty acid oxidation and is therefore elevated levels of HEL are indicative of excessive ROS in an individual. HEL concentration in human urine has been reported to be 22.9 nmol/L. See Sakai, K., et al., “Determination of HEL (hexanoyl-lysine adduct): a novel biomarker for omega-6 PUFA oxidation,” Subcell Biochem., 77, 61-72, (2014). In various embodiments, antioxidant capacity testing employs a CUPRAC (cupric reducing antioxidant capacity) method for measuring the sum of the antioxidant activity due to multiple species (uric acid, proteins, vitamins, dietary supplements) present in a urine sample (See e.g., Ozyurek, M., Guclu, K. and Apak, R., “The main and modified CUPRAC methods of antioxidant measurement,” Trends in Analytical Chemistry, 30: 652-664 (2011)). Alternatively, or additionally, modified methods can be used to specifically measure or to discriminate among uric acid, ascorbic proteins or other substances that contribute to the overall antioxidant capacity, thereby monitoring what is referred to as the “antioxidant reserve.” Several other biomarkers can be used to gauge antioxidant capacity and non-limiting examples are listed in TABLE 1 above. The CUPRAC method, and other methods that employ a redox indicator, directly measure the reaction of antioxidants with substances having the appropriate redox potential to effect a visible color change or a color interpretable by a simple colorimeter. A higher value for antioxidant power, that is, a greater level of biomarkers indicative of antioxidant capacity, indicates a healthy individual because the individual has compounds that can neutralize free radicals that cause oxidative damage and stress.

Certain embodiments commensurate in scope with the originally claimed invention are summarized below. These embodiments are not intended to limit the scope of the claimed invention, but rather these embodiments are intended only to provide a brief summary of possible forms of the invention. Indeed, the invention may encompass a variety of forms that may be similar to or different from the embodiments set forth below. One or more specific embodiments of the present subject matter will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The term “platelet plasma” as used herein refers to platelet rich plasma (PRP), human platelet lysate (HPL), and combinations thereof.

“Platelet rich plasma” (PRP) as described herein is a blood plasma that has been enriched with platelets. As a concentrated source of autologous platelets, PRP contains and releases several different growth factors and other cytokines that stimulate healing of bone and soft tissue. Components of PRP may include but are not limited to platelet-derived growth factor, transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, and combinations thereof.

PRP may be prepared by collection of the patient's whole blood (that is anticoagulated with citrate dextrose) before undergoing two stages of centrifugation designed to separate the PRP aliquot from platelet-poor plasma and red blood cells. In humans, a typical baseline blood platelet count may range from about 150,000 to about 450,000 platelets per .mu.l of blood, or about 200,000 platelets per .mu.l of blood. Therapeutic PRP may concentrate platelets in plasma by about five-fold. As such, PRP platelet count in PRP may range from about 750,000 to about 2.25.times.10.sup.6 platelets per .mu.l of PRP, or about 1.times.0.10.sup.6 platelets per .mu.l of blood. The PRP may then be used to prepare human platelet lysate.

Compositions of the present disclosure may comprise platelet plasma compositions from PRP, HPL, or combinations thereof, and either platelet plasma composition may be used to regenerate ovarian tissue for augmentation of fertility. Further, the platelet plasma composition may be used with or without concentrated bone marrow (BMAC). By way of example, when administered into ovarian tissue, about 0.05 to about 2.0 cc of platelet plasma composition may be used. Platelets are non-nucleated blood cells that as noted above are found in bone marrow and peripheral blood.

In various embodiments of the present invention, the platelet plasma composition may be obtained by sequestering platelets from whole blood or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires ovarian tissue regeneration. In some embodiments the lysate will act more rapidly than the PRP (or platelet poor plasma from bone marrow).

Human platelet lysate may be formed from but not limited to PRP, pooled platelets from humans, and cultured megakaryocytes from stem cell expansion technology. In some embodiments, HPL is from a commercial source. In some embodiments, HPL is prepared in the laboratory from platelet rich plasma (PRP), pooled platelets from humans, or cultured megakaryocytes from stem cell expansion technology.

Notably, platelet poor plasma that is derived from bone marrow has a greater platelet concentration than platelet rich plasma from blood, also known as platelet poor/rich plasma (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow.) In various embodiments, the platelet plasma composition, which may or may not be in the form of a lysate, may serve one or more of the following functions: (1) to release/provide growth factors and cytokines for tissue regeneration; (2) to reduce inflammation; (3) to attract/mobilize cell signaling; (4) to initiate repair of damaged/atrophied ovarian tissue through fibroblast growth factors (FGF); (5) to stabilize extracellular matrix in the ovary; (6) to stimulate maturation of immature oocytes; (7) to stimulate revascularization of fibrotic tissue; and (8) to stimulate oocyte receptivity to spermatozoa. Additionally, by combining platelet therapy with stem cells, there can be synergy with respect to augmentation of fertility.

In some embodiments in which the lysate is used, the cytokines may be concentrated in order to optimize their functional capacity. Concentration may be accomplished in two steps. First, blood may be obtained and concentrated to a volume that is 5-15% of what it was before concentration. Devices that may be used include but are not limited to a hemofilter or a hemo-concentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing 20-500 cc of peripheral blood, 40-250 cc of peripheral blood, or 60-100 cc of peripheral blood. The amount of blood that one should draw will depend on the extent of ovarian tissue degeneration.

In some embodiments, a method of generation of said PRP may be used according to U.S. Pat. No. 9,011,929, which is incorporated by reference herein in its entirety. In essence, a method may comprise separating PRP from whole blood by collecting whole blood from an animal or patient into a vacuum test tube containing sodium citrate, and primarily centrifuging the collected whole blood; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid; and collecting the PRP concentrated in a bottom layer by another blunt needle; mixing the PRP collected from the separating step with a calcium chloride solution by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at room temperature before mixing; and mixing the mixture of the PRP and the calcium chloride solution with the type I collagen, in an opaque phase, four times by another three-way connector.

In an exemplary embodiment of the disclosure, a method may comprise separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector.

The term “platelet-rich plasma” or “PRP” as used herein is a broad term which is used in its ordinary sense and is a concentration of platelets greater than the peripheral blood concentration suspended in a solution of plasma, or other excipient suitable for administration to a human or non-human animal including, but not limited to, isotonic sodium chloride solution, physiological saline, normal saline, dextrose 5% in water, dextrose 10% in water, Ringer solution, lactated Ringer solution, Ringer lactate, Ringer lactate solution, and the like. PRP compositions may be an autologous preparation from whole blood taken from the subject to be treated or, alternatively, PRP compositions may be prepared from a whole blood sample taken from a single donor source or from whole blood samples taken from multiple donor sources. In general, PRP compositions comprise platelets at a platelet concentration that is higher than the baseline concentration of the platelets in whole blood. In some embodiments, PRP compositions may further comprise WBCs at a WBC concentration that is higher than the baseline concentration of the WBCs in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration. In some embodiments, PRP compositions comprise elevated concentrations of platelets and WBCs and lower levels of RBCs and hemoglobin relative to their baseline concentrations. In some embodiments of PRP composition, only the concentration of platelets is elevated relative to the baseline concentration. Some embodiments of PRP composition comprise elevated levels of platelets and WBCs compared to baseline concentrations. In some embodiments, PRP compositions comprise elevated concentrations of platelets and lower levels of neutrophils relative to their baseline concentrations. Some embodiments of PRP composition comprise elevated levels of platelets and neutrophil-depleted WBCs compared to their baseline concentrations. In some embodiments of PRP, the ratio of lymphocytes and monocytes to neutrophils is significantly higher than the ratios of their baseline concentrations. The PRP formulation may include platelets at a level of between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood comprises at least 140,000 to 150,000 platelets and up to 400,000 to 500,000 platelets. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter. The WBC concentration is typically elevated in PRP compositions. For example, the WBC concentration may be between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000; about 10,000 and about 15,000; about 15,000 and about 20,000; about 20,000 and about 30,000; about 30,000 and about 50,000; about 50,000 and about 75,000; and about 75,000 and about 100,000. Among the WBCs in the PRP composition, the concentrations may vary by the cell type but, generally, each may be elevated. In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration. The concentrations of eosinophils in the PRP composition may be less than baseline, about 1.5 times baseline, about 2 times baseline, about 3 times baseline, about 5 times baseline, or higher.

In whole blood, the lymphocyte count is typically between 1,300 and 4,000 cells per microliter, but in other examples, the lymphocyte concentration may be between about 5,000 and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter.

In certain variations, the PRP composition may contain a specific concentration of neutrophils. The neutrophil concentration may vary between less than the baseline concentration of neutrophils to eight times than the baseline concentration of neutrophils. In some embodiments, the PRP composition may include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels are depleted to 1% or less than that found in whole blood. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, about 5,000 to about 20,000 per microliter, about 20,000 to about 40,000 per microliter, or about 40,000 to about 60,000 per microliter. In some embodiments, neutrophils are eliminated or substantially eliminated. Means to deplete blood products, such as PRP, of neutrophils is known and discussed in U.S. Pat. No. 7,462,268, which is incorporated herein by reference. Several embodiments are directed to PRP compositions in which levels of platelets and white blood cells are elevated compared to whole blood and in which the ratio of monocytes and/or lymphocytes to neutrophils is higher than in whole blood. The ratio of monocytes and/or lymphocytes to neutrophils may serve as an index to determine if a PRP formulation may be efficaciously used as a treatment for a particular disease or condition. PRP compositions where the ratio of monocytes and/or lymphocytes to neutrophils is increased may be generated by either lowering neutrophils levels, or by maintaining neutrophil levels while increasing levels of monocytes and/or lymphocytes. Several embodiments relate to a PRP formulation that contains 1.01 times, or higher, baseline platelets in combination with a 1.01 times, or higher, baseline white blood cells with the neutrophil component depleted at least 1% from baseline. In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women. In some embodiments, PRP compositions contain increased concentrations of growth factors and other cytokines. In several embodiments, PRP compositions may include increased concentrations of one or more of: platelet-derived growth factor, transforming growth factor beta, fibroblast growth factor, insulin-like growth factor, insulin-like growth factor 2, vascular endothelial growth factor, epidermal growth factor, interleukin-8, keratinocyte growth factor, and connective tissue growth factor. In some embodiments, the platelets collected in PRP are activated by thrombin and calcium chloride to induce the release of these growth factors from alpha granules. In some embodiments, a PRP composition is activated exogenously with thrombin and/or calcium to produce a gel that can be applied to an area to be treated. The process of exogenous activation, however, results in immediate release of growth factors. Other embodiments relate to activation of PRP via in vivo contact with collagen containing tissue at the treatment site. The in vivo activation of PRP results in slower growth factor release at the desired site.

In certain embodiments of the invention, the PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the tissue damage and/or the MI, the availability of prepared PRP, or any suitable combination of factors. Any suitable amount of blood may be collected. For example, about 1 cc to about 150 cc of blood or more may be drawn. More specifically, about 27 cc to about 110 cc or about 27 cc to about 55 cc of blood may be withdrawn. In some embodiments, the blood may be collected from a patient who may be presently suffering, or who has previously suffered from, connective tissue damage and/or an MI. PRP made from a patient's own blood may significantly reduce the risk of adverse reactions or infection.

In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area. The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume.

In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained.

As the PRP composition comprises activated platelets, active agents within the platelets are released. These agents include, but are not limited to, cytokines (e.g., IL-1B, IL-6, TNF-A), chemokines (e.g., ENA-78 (CXCL8), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-.beta. 1, 2, and 3, and VEGF).

Said PRP may be used to treat autologous regenerative cells prior to administration of said cells for stimulation of ovary regeneration and/or prevention of immunologically mediated abortions. One type of autologous regenerative cells are adipose stromal vascular fraction cells. Said stromal vascular fraction cells are obtained by the following steps; a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37.degree. C. shaking water bath for one hour then centrifuged for 5 min at 300 rcf; g) Following centrifugation, the stromal cells are resuspended within Isolyte in separate sterile 50 mL centrifuge tubes; h) The tubes are centrifuged for 5 min. at 300 rcf and the Isolyte is removed, leaving cell pellet; i) The pellets are resuspended in 40 ml of Isolyte, centrifuged again for 5 min at 300rcf. The supernatant is again be removed; j) The cell pellets are combined and filtered through 100.quadrature.m cell strainers into a sterile 50 ml centrifuge tube and centrifuged for 5 min at 300rcf and the supernatant removed, leaving the pelleted adipose stromal cells. Means of combining PRP and SVF are known in the literature and incorporated by reference [3-7].

In some embodiments, the neutrophils are depleted by at least 5%, in some embodiments, the neutrophils are depleted by at least 10%, in some embodiments, the neutrophils are depleted by at least 15%, in some embodiments, the neutrophils are depleted by at least 20%, in some embodiments, the neutrophils are depleted by at least 25%, in some embodiments, the neutrophils are depleted by at least 30%, in some embodiments, the neutrophils are depleted by at least 35%, in some embodiments, the neutrophils are depleted by at least 40%, in some embodiments, the neutrophils are depleted by at least 45%, in some embodiments, the neutrophils are depleted by at least 50%, in some embodiments, the neutrophils are depleted by at least 55%, in some embodiments, the neutrophils are depleted by at least 60%, in some embodiments, the neutrophils are depleted by at least 65%, in some embodiments, the neutrophils are depleted by at least 70%, in some embodiments, the neutrophils are depleted by at least 75%, in some embodiments, the neutrophils are depleted by at least 80%, in some embodiments, the neutrophils are depleted by at least 85%, in some embodiments, the neutrophils are depleted by at least 90%, in some embodiments, the neutrophils are depleted by at least 95%, in some embodiments, the neutrophils are depleted by at least 95%. In some embodiments, the neutrophils in the platelet rich plasma are substantially removed.

### Example 1: Reduction of Brain Microglial Activation by PRP and Pterostilbene

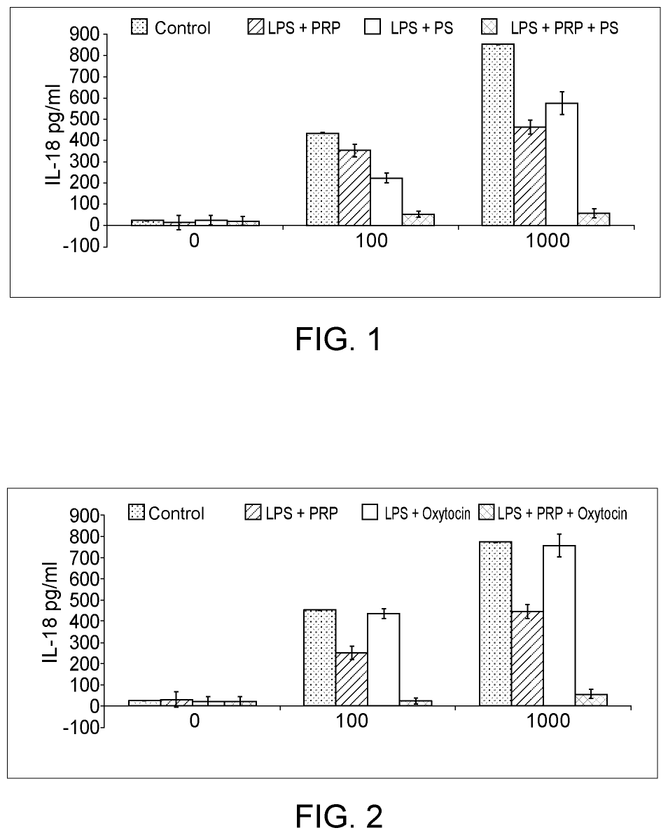

BALB/c mice were anesthetized with isofluorane and administered pterstilbene (0.4 mg/mouse) and/or platelet rich plasma (5 microliter per mouse) via intranasal route subsequent to induction of systemic inflammation by intraperitoneal administration of endotoxin. Mice were sacrificed after 24 hours of treatment and quantification of cytokine IL-18 was performed by ELISA from brain homogenate tissue. As seen below, a synergistic reduction of TNF-alpha was observed. Results are shown in FIG. 1.

### Example 2: Reduction of Brain Microglial Activation by PRP and Oxytocin

BALB/c mice were anesthetized with isofluorane and administered oxytocin (0.1 IU/mouse) and/or platelet rich plasma (5 microliter per mouse) via intranasal route subsequent to induction of systemic inflammation by intraperitoneal administration of endotoxin. Mice were sacrificed after 24 hours of treatment and quantification of cytokine IL-18 was performed by ELISA from brain homogenate tissue. As seen below, a synergistic reduction of TNF-alpha was observed. Results are shown in FIG. 2.

### REFERENCES

1. Brynskikh, A., et al., Adaptive immunity affects learning behavior in mice. Brain Behav Immun, 2008. 22(6): p. 861-9.
2. Derecki, N. C., K. M. Quinnies, and J. Kipnis, Alternatively activated myeloid (M2) cells enhance cognitive function in immune compromised mice. Brain Behav Immun, 2011. 25(3): p. 379-85.
3. Lewitus, G. M., H. Cohen, and M. Schwartz, Reducing post-traumatic anxiety by immunization. Brain Behav Immun, 2008. 22(7): p. 1108-1114.
4. Clark, S. M., et al., CD4(+) T cells confer anxiolytic and antidepressant-like effects, but enhance fear memory processes in Rag2(−/−) mice. Stress, 2016. 19(3): p. 303-11.
5. Clark, S. M., et al., Neonatal adoptive transfer of lymphocytes rescues social behaviour during adolescence in immune-deficient mice. Eur J Neurosci, 2018. 47(8): p. 968-978.
6. Radjavi, A., I. Smirnov, and J. Kipnis, Brain antigen-reactive CD4+ T cells are sufficient to support learning behavior in mice with limited T cell repertoire. Brain Behav Immun, 2014. 35: p. 58-63.
7. Horbelt, T., et al., Behaviorally conditioned immunosuppression with cyclosporine A forms long lasting memory trace. Behav Brain Res, 2019. 376: p. 112208.
8. Luckemann, L., et al., Behavioral conditioning of anti-proliferative and immunosuppressive properties of the mTOR inhibitor rapamycin. Brain Behav Immun, 2019. 79: p. 326-331.
9. Kirchhof, J., et al., Learned immunosuppressive placebo responses in renal transplant patients. Proc Natl Acad Sci USA, 2018. 115(16): p. 4223-4227.
10. Wirth, T., et al., Repeated recall of learned immunosuppression: evidence from rats and men. Brain Behav Immun, 2011. 25(7): p. 1444-51.
11. Goebel, M. U., et al., Behavioral conditioning of immunosuppression is possible in humans. FASEB J, 2002. 16(14): p. 1869-73.
12. Roudebush, R. E. and H. U. Bryant, Conditioned immunosuppression of a murine delayed type hypersensitivity response: dissociation from corticosterone elevation. Brain Behav Immun, 1991. 5(3): p. 308-17.
13. MacQueen, G. M. and S. Siegel, Conditional immunomodulation following training with cyclophosphamide. Behav Neurosci, 1989. 103(3): p. 638-47.
14. Kusnecov, A. W., A. J. Husband, and M. G. King, Behaviorally conditioned suppression of mitogen-induced proliferation and immunoglobulin production: effect of time span between conditioning and reexposure to the conditioning stimulus. Brain Behav Immun, 1988. 2(3): p. 198-211.
15. Lysle, D. T., et al., Pavlovian conditioning of shock-induced suppression of lymphocyte reactivity: acquisition, extinction, and preexposure effects. Life Sci, 1988. 42(22): p. 2185-94.
16. Gorczynski, R. M., Analysis of lymphocytes in, and host environment of, mice showing conditioned immunosuppression to cyclophosphamide. Brain Behav Immun, 1987. 1(1): p. 21-35.
17. Janz, L. J., et al., Pavlovian conditioning of LPS-induced responses: effects on corticosterone, splenic NE, and IL-2 production. Physiol Behav, 1996. 59(6): p. 1103-9.
18. Gorczynski, R. M., Toward an understanding of the mechanisms of classical conditioning of antibody responses. J Gerontol, 1991. 46(4): p. P152-6.
19. Kusnecov, A., M. G. King, and A. J. Husband, Immunomodulation by behavioural conditioning. Biol Psychol, 1989. 28(1): p. 25-39.
20. Hiramoto, R. N., et al., Regulation of natural immunity (NK activity) by conditioning. Ann N Y Acad Sci, 1987. 496: p. 545-52.
21. Neveu, P. J., R. Dantzer, and M. Le Moal, Behaviorally conditioned suppression of mitogen-induced lymphoproliferation and antibody production in mice. Neurosci Lett, 1986. 65(3): p. 293-8.
22. Bauer, D., et al., Behavioral Conditioning of Immune Responses with Cyclosporine A in a Murine Model of Experimental Autoimmune Uveitis. Neuroimmunomodulation, 2017. 24(2): p. 87-99.
23. Lysle, D. T., L. J. Luecken, and K. A. Maslonek, Suppression of the development of adjuvant arthritis by a conditioned aversive stimulus. Brain Behav Immun, 1992. 6(1): p. 64-73.
24. Blom, J. M., et al., Learned immunosuppression is associated with an increased risk of chemically-induced tumors. Neuroimmunomodulation, 1995. 2(2): p. 92-9.
25. Luckemann, L., et al., Learned Immunosuppressive Placebo Response Attenuates Disease Progression in a Rodent Model of Rheumatoid Arthritis. Arthritis Rheumatol, 2020. 72(4): p. 588-597.
26. Pacheco-Lopez, G., et al., Calcineurin inhibition in splenocytes induced by pavlovian conditioning. FASEB J, 2009. 23(4): p. 1161-7.
27. Hsueh, C. M., et al., Involvement of catecholamines in recall of the conditioned NK cell response. J Neuroimmunol, 1999. 94(1-2): p. 172-81.
28. Coussons-Read, M. E., L. A. Dykstra, and D. T. Lysle, Pavlovian conditioning of morphine-induced alterations of immune status: evidence for peripheral beta-adrenergic receptor involvement. Brain Behav Immun, 1994. 8(3): p. 204-17.
29. Saba, F., et al., G-CSF induces up-regulation of CXCR4 expression in human hematopoietic stem cells by beta-adrenergic agonist. Hematology, 2015. 20(8): p. 462-468.
30. Pasupuleti, L. V., et al., Do all beta-blockers attenuate the excess hematopoietic progenitor cell mobilization from the bone marrow following trauma/hemorrhagic shock? J Trauma Acute Care Surg, 2014. 76(4): p. 970-5.
31. Chen, C., et al., Adrenaline administration promotes the efficiency of granulocyte colony stimulating factor-mediated hematopoietic stem and progenitor cell mobilization in mice. Int J Hematol, 2013. 97(1): p. 50-7.
32. Spiegel, A., et al., Catecholaminergic neurotransmitters regulate migration and repopulation of immature human CD34+ cells through Wnt signaling. Nat Immunol, 2007. 8(10): p. 1123-31.
33. Katayama, Y., et al., Signals from the sympathetic nervous system regulate hematopoietic stem cell egress from bone marrow. Cell, 2006. 124(2): p. 407-21.
34. Kose, S., et al., Human bone marrow mesenchymal stem cells secrete endocannabinoids that stimulate in vitro hematopoietic stem cell migration effectively comparable to beta-adrenergic stimulation. Exp Hematol, 2018. 57: p. 30-41 el.
35. Wang, H., et al., Nicotinic acetylcholine receptor alpha7 subunit is an essential regulator of inflammation. Nature, 2003. 421(6921): p. 384-8.
36. Tracey, K. J., The inflammatory reflex. Nature, 2002. 420(6917): p. 853-9.
37. Borovikova, L. V., et al., Role of vagus nerve signaling in CNI-1493-mediated suppression of acute inflammation. Auton Neurosci, 2000. 85(1-3): p. 141-7.
38. Borovikova, L. V., et al., Vagus nerve stimulation attenuates the systemic inflammatory response to endotoxin. Nature, 2000. 405(6785): p. 458-62.
39. Koopman, F. A., et al., Vagus nerve stimulation inhibits cytokine production and attenuates disease severity in rheumatoid arthritis. Proc Natl Acad Sci USA, 2016. 113(29): p. 8284-9.
40. Chao, C. C., et al., Priming effect of morphine on the production of tumor necrosis factor-alpha by microglia: implications in respiratory burst activity and human immunodeficiency virus-1 expression. J Pharmacol Exp Ther, 1994. 269(1): p. 198-203.
41. Peterson, P. K., et al., Morphine stimulates phagocytosis of Mycobacterium tuberculosis by human microglial cells: involvement of a G protein-coupled opiate receptor. Adv Neuroimmunol, 1995. 5(3): p. 299-309.
42. Liu, Y., et al., Morphine stimulates nitric oxide release from invertebrate microglia. Brain Res, 1996. 722(1-2): p. 125-31.
43. Magazine, H. I., et al., Morphine-induced conformational changes in human monocytes, granulocytes, and endothelial cells and in invertebrate immunocytes and microglia are mediated by nitric oxide. J Immunol, 1996. 156(12): p. 4845-50.
44. Lipovsky, M. M., et al., Morphine enhances complement receptor-mediated phagocytosis of Cryptococcus neoformans by human microglia. Clin Immunol Immunopathol, 1998. 87(2): p. 163-7.
45. Cui, Y., et al., Activation of p38 mitogen-activated protein kinase in spinal microglia mediates morphine antinociceptive tolerance. Brain Res, 2006. 1069(1): p. 235-43.
46. Qian, L., et al., Microglia-mediated neurotoxicity is inhibited by morphine through an opioid receptor-independent reduction of NADPH oxidase activity. J Immunol, 2007. 179(2): p. 1198-209.
47. Cui, Y., et al., A novel role of minocycline: attenuating morphine antinociceptive tolerance by inhibition of p38 MAPK in the activated spinal microglia. Brain Behav Immun, 2008. 22(1): p. 114-23.
48. Mika, J., Modulation of microglia can attenuate neuropathic pain symptoms and enhance morphine effectiveness. Pharmacol Rep, 2008. 60(3): p. 297-307.
49. Mika, J., et al., Importance of glial activation in neuropathic pain. Eur J Pharmacol, 2013. 716(1-3): p. 106-19.
50. Arruda, J. L., et al., Intrathecal anti-IL-6 antibody and IgG attenuates peripheral nerve injury-induced mechanical allodynia in the rat: possible immune modulation in neuropathic pain. Brain Res, 2000. 879(1-2): p. 216-25.
51. Zalcman, S., I. Savina, and R. A. Wise, Interleukin-6 increases sensitivity to the locomotor-stimulating effects of amphetamine in rats. Brain Res, 1999. 847(2): p. 276-83.
52. Nicolaou, C., et al., Serum cytokine concentrations in alcohol-dependent individuals without liver disease. Alcohol, 2004. 32(3): p. 243-7.
53. Ersche, K. D., et al., Aberrant disgust responses and immune reactivity in cocaine-dependent men. Biol Psychiatry, 2014. 75(2): p. 140-7.
54. Kane, C. J., et al., Effects of ethanol on immune response in the brain: region-specific changes in adolescent versus adult mice. Alcohol Clin Exp Res, 2014. 38(2): p. 384-91.
55. Fuster, D., et al., Inflammatory cytokines and mortality in a cohort of HIV-infected adults with alcohol problems. AIDS, 2014. 28(7): p. 1059-64.
56. Dahl, J., et al., The plasma levels of various cytokines are increased during ongoing depression and are reduced to normal levels after recovery. Psychoneuroendocrinology, 2014. 45: p. 77-86.
57. Neupane, S. P., et al., High frequency and intensity of drinking may attenuate increased inflammatory cytokine levels of major depression in alcohol-use disorders. CNS Neurosci Ther, 2014. 20(10): p. 898-904.
58. Chan, Y. Y., et al., Inflammatory response in heroin addicts undergoing methadone maintenance treatment. Psychiatry Res, 2015. 226(1): p. 230-4.
59. Heberlein, A., et al., TNF-alpha and IL-6 serum levels: neurobiological markers of alcohol consumption in alcohol-dependent patients? Alcohol, 2014. 48(7): p. 671-6.
60. Kaplan, M. S. and J. W. Hinds, Neurogenesis in the adult rat: electron microscopic analysis of light radioautographs. Science, 1977. 197(4308): p. 1092-4.
61. Lubbers, K., J. R. Wolff, and M. Frotscher, Neurogenesis of GABAergic neurons in the rat dentate gyrus: a combined autoradiographic and immunocytochemical study. Neurosci Lett, 1985. 62(3): p. 317-22.
62. Eriksson, P. S., et al., Neurogenesis in the adult human hippocampus. Nat Med, 1998. 4(11): p. 1313-7.
63. Roy, N. S., et al., In vitro neurogenesis by progenitor cells isolated from the adult human hippocampus. Nat Med, 2000. 6(3): p. 271-7.
64. Hauser, K. F., et al., Opioids intrinsically inhibit the genesis of mouse cerebellar granule neuron precursors in vitro: differential impact of mu and delta receptor activation on proliferation and neurite elongation. Eur J Neurosci, 2000. 12(4): p. 1281-93.
65. Eisch, A. J. and C. D. Mandyam, Drug dependence and addiction, II: Adult neurogenesis and drug abuse. Am J Psychiatry, 2004. 161(3): p. 426.
66. Fischer, S. J., et al., Morphine blood levels, dependence, and regulation of hippocampal subgranular zone proliferation rely on administration paradigm. Neuroscience, 2008. 151(4): p. 1217-24.
67. Cunha-Oliveira, T., A. C. Rego, and C. R. Oliveira, Cellular and molecular mechanisms involved in the neurotoxicity of opioid and psychostimulant drugs. Brain Res Rev, 2008. 58(1): p. 192-208.
68. Jin, J., et al., Interaction of the mu-opioid receptor with GPR177 (Wntless) inhibits Wnt secretion: potential implications for opioid dependence. BMC Neurosci, 2010. 11: p. 33.
69. Zheng, H., P. Y. Law, and H. H. Loh, Non-Coding RNAs Regulating Morphine Function: With Emphasis on the In vivo and In vitro Functions of miR-190. Front Genet, 2012. 3: p. 113.
70. Hafizi, M., et al., Exploring the enkephalinergic differentiation potential in adult stem cells for cell therapy and drug screening implications. In Vitro Cell Dev Biol Anim, 2012. 48(9): p. 562-9.
71. Zheng, H., et al., NeuroD modulates opioid agonist-selective regulation of adult neurogenesis and contextual memory extinction. Neuropsychopharmacology, 2013. 38(5): p. 770-7.
72. Bernstein, H. G., et al., Increased densities of nitric oxide synthase expressing neurons in the temporal cortex and the hypothalamic paraventricular nucleus of polytoxicomanic heroin overdose victims: possible implications for heroin neurotoxicity. Acta Histochem, 2014. 116(1): p. 182-90.
73. Teuchert-Noodt, G., R. R. Dawirs, and K. Hildebrandt, Adult treatment with methamphetamine transiently decreases dentate granule cell proliferation in the gerbil hippocampus. J Neural Transm (Vienna), 2000. 107(2): p. 133-43.
74. Yamaguchi, M., et al., Repetitive cocaine administration decreases neurogenesis in adult rat hippocampus. Ann N Y Acad Sci, 2004. 1025: p. 351-62.
75. Kazma, M., et al., Survival, differentiation, and reversal of heroin neurobehavioral teratogenicity in mice by transplanted neural stem cells derived from embryonic stem cells. J Neurosci Res, 2010. 88(2): p. 315-23.
76. Rhodin, A., et al., Recombinant human growth hormone improves cognitive capacity in a pain patient exposed to chronic opioids. Acta Anaesthesiol Scand, 2014. 58(6): p. 759-65.
77. Cameron, H. A. and E. Gould, Adult neurogenesis is regulated by adrenal steroids in the dentate gyrus. Neuroscience, 1994. 61(2): p. 203-9.
78. Montaron, M. F., et al., Adrenalectomy increases neurogenesis but not PSA-NCAM expression in aged dentate gyrus. Eur J Neurosci, 1999. 11(4): p. 1479-85.
79. Brezun, J. M. and A. Daszuta, Depletion in serotonin decreases neurogenesis in the dentate gyrus and the subventricular zone of adult rats. Neuroscience, 1999. 89(4): p. 999-1002.
80. Gould, E., Serotonin and hippocampal neurogenesis. Neuropsychopharmacology, 1999. 21(2 Suppl): p. 46S-51S.
81. Brezun, J. M. and A. Daszuta, Serotonin may stimulate granule cell proliferation in the adult hippocampus, as observed in rats grafted with foetal raphe neurons. Eur J Neurosci, 2000. 12(1): p. 391-6.
82. Wagner, J. P., I. B. Black, and E. DiCicco-Bloom, Stimulation of neonatal and adult brain neurogenesis by subcutaneous injection of basic fibroblast growth factor. J Neurosci, 1999. 19(14): p. 6006-16.
83. Palmer, T. D., et al., Fibroblast growth factor-2 activates a latent neurogenic program in neural stem cells from diverse regions of the adult CNS. J Neurosci, 1999. 19(19): p. 8487-97.
84. Malberg, J. E., et al., Chronic antidepressant treatment increases neurogenesis in adult rat hippocampus. J Neurosci, 2000. 20(24): p. 9104-10.
85. Madsen, T. M., et al., Increased neurogenesis in a model of electroconvulsive therapy. Biol Psychiatry, 2000. 47(12): p. 1043-9.
86. Chen, G., et al., Enhancement of hippocampal neurogenesis by lithium. J Neurochem, 2000. 75(4): p. 1729-34.
87. O'Kusky, J. R., P. Ye, and A. J. D'Ercole, Insulin-like growth factor-I promotes neurogenesis and synaptogenesis in the hippocampal dentate gyrus during postnatal development. J Neurosci, 2000. 20(22): p. 8435-42.
88. Vallieres, L., et al., Reduced hippocampal neurogenesis in adult transgenic mice with chronic astrocytic production of interleukin-6. J Neurosci, 2002. 22(2): p. 486-92.
89. Jin, K., et al., Heparin-binding epidermal growth factor-like growth factor: hypoxia-inducible expression in vitro and stimulation of neurogenesis in vitro and in vivo. J Neurosci, 2002. 22(13): p. 5365-73.
90. Jin, K., et al., Vascular endothelial growth factor (VEGF) stimulates neurogenesis in vitro and in vivo. Proc Natl Acad Sci USA, 2002. 99(18): p. 11946-50.
91. Karishma, K. K. and J. Herbert, Dehydroepiandrosterone (DHEA) stimulates neurogenesis in the hippocampus of the rat, promotes survival of newly formed neurons and prevents corticosterone-induced suppression. Eur J Neurosci, 2002. 16(3): p. 445-53.
92. Lee, J., W. Duan, and M. P. Mattson, Evidence that brain-derived neurotrophic factor is required for basal neurogenesis and mediates, in part, the enhancement of neurogenesis by dietary restriction in the hippocampus of adult mice. J Neurochem, 2002. 82(6): p. 1367-75.
93. Nacher, J., et al., NMDA receptor antagonist treatment increases the production of new neurons in the aged rat hippocampus. Neurobiol Aging, 2003. 24(2): p. 273-84.
94. Uchida, K., et al., Stimulatory effects of prostaglandin E2 on neurogenesis in the dentate gyrus of the adult rat. Zoolog Sci, 2002. 19(11): p. 1211-6.
95. Shingo, T., et al., Pregnancy-stimulated neurogenesis in the adult female forebrain mediated by prolactin. Science, 2003. 299(5603): p. 117-20.
96. Bal, F., M. Bergeron, and D. L. Nelson, Chronic AMPA receptor potentiator (LY451646) treatment increases cell proliferation in adult rat hippocampus. Neuropharmacology, 2003. 44(8): p. 1013-21.
97. Mercer, A., et al., PACAP promotes neural stem cell proliferation in adult mouse brain. J Neurosci Res, 2004. 76(2): p. 205-15.
98. Kim, J. S., et al., Lithium selectively increases neuronal differentiation of hippocampal neural progenitor cells both in vitro and in vivo. J Neurochem, 2004. 89(2): p. 324-36.
99. Arias-Carrion, O., et al., Neurogenesis in the subventricular zone following transcranial magnetic field stimulation and nigrostriatal lesions. J Neurosci Res, 2004. 78(1): p. 16-28.
100. Kodama, M., T. Fujioka, and R. S. Duman, Chronic olanzapine or fluoxetine administration increases cell proliferation in hippocampus and prefrontal cortex of adult rat. Biol Psychiatry, 2004. 56(8): p. 570-80.
101. Herrera, D. G., et al., Selective impairment of hippocampal neurogenesis by chronic alcoholism: protective effects of an antioxidant. Proc Natl Acad Sci USA, 2003. 100(13): p. 7919-24.

1. A method of reducing opioid addiction comprising the steps of:
a) identifying a patient with a propensity for opioid addiction;
b) assessing one or more markers associated with said opioid addiction; and
c) administering to said patient platelet rich plasma and/or cord blood plasma alone or in combination with an anti-inflammatory/regenerative adjuvant;

2. The method of claim 1, wherein propensity for opioid addiction is associated with augmentation of inflammatory cytokines and/or mediators in a biological fluid as compared to an age-matched control.

3. The method of claim 2, wherein said biological fluid is blood, plasma, or serum.

4. The method of claim 2, wherein said biological fluid is urine.

5. The method of claim 2, wherein said biological fluid is saliva.

6. The method of claim 2, wherein said biological fluid is tears.

7. The method of claim 2, wherein said biological fluid is bronchiolar lavage fluid.

8. The method of claim 2, wherein said biological fluid is cerebral spinal fluid.

9. The method of claim 2, wherein said inflammatory cytokine is IL-1 beta.

10. The method of claim 2, wherein said inflammatory cytokine is IL-6.

11. The method of claim 2, wherein said inflammatory cytokine is IL-7.

12. The method of claim 2, wherein said inflammatory cytokine is IL-8.

13. The method of claim 2, wherein said inflammatory cytokine is IL-12.

14. The method of claim 2, wherein said inflammatory cytokine is IL-15.

15. The method of claim 2, wherein said inflammatory cytokine is IL-17.

16. The method of claim 2, wherein said inflammatory cytokine is IL-18.

17. The method of claim 2, wherein said inflammatory cytokine is IL-21.

18. The method of claim 2, wherein said inflammatory cytokine is IL-9.

19. The method of claim 2, wherein said inflammatory cytokine is IL-27.

20. The method of claim 2, wherein said inflammatory cytokine is IL-23.
